(19) United States
(12) Patent Application Publication
(10) Pub. No.: US 2024/0293606 A1
(43) Pub. Date: Sep. 5, 2024

(54) SYSTEM AND METHOD FOR TREATING A GASTROINTESTINAL TRACT OF A SUBJECT

(71) Applicant: VACURE Ltd., Ra'anana (IL)

(72) Inventors: Michael Bortz, Kfar Saba (IL); Adam Sagiv, Bnai Atarot (IL); Oded Meiri, Ram On (IL); Petros Benias, Englewood Cliffs, NJ (US); Mitchell Roslin, Armonk, NY (US)

(21) Appl. No.: 18/418,514

(22) Filed: Jan. 22, 2024

Related U.S. Application Data
(60) Provisional application No. 63/440,115, filed on Jan. 20, 2023.

(51) Int. Cl. A61M 1/00

(52) U.S. Cl. CPC A61M 1/84; A61M 2205/0216; A61M 2205/0266; A61M 2210/1042

(57) ABSTRACT

A medical system for applying negative pressure within a gastrointestinal tract of a subject. The medical system includes an elongate tube defining at least one interior channel, a plurality of orifices in fluid communication with the interior channel, and at least one exterior channel. The exterior channel is in fluid communication with the interior channel via at least one of the orifices. The elongate tube has a delivery state, and a first operative state in which the elongate tube forms a coil including a plurality of loops. A fluid-tight lumen is in fluid communication with the elongate tube. The lumen is adapted to couple to a source of negative pressure for delivery of negative pressure to the elongate tube. In the first operative state of the elongate tube, a length of the coil is at least 15 mm and the plurality of loops includes at least 4 loops.

### CROSS-REFERENCE TO RELATED APPLICATIONS

This patent application claims the benefit of U.S. Provisional Patent Application No. 63/440,115 filed on Jan. 20, 2023, which is incorporated herein by reference in its entirety.

### BACKGROUND

Wounds in the gastrointestinal tract such as perforations and post-surgical leaks, and particularly in the esophagus, are common in endoscopic and open surgical procedures. The endoluminal location of these wounds and natural wet environment surrounding the wounds make these wounds particularly difficult to treat. Limited treatment options exist for these wounds which have significant morbidity and mortality rates or involve extensive hospital stay.

Vacuum assisted closure (VAC) therapy can increase the rate of wound closure. Negative pressure wound therapy (NPWT) or VAC therapy is the application of sub-atmospheric pressure to acute or chronic wounds to promote the healing of a wound. In theory, creating a negative-pressure in the local wound environment, draws away bacteria, exudate, fluid, and debris tissue from the wound site, increases the rate of healing by promoting blood flow and facilitates localized cell migration and proliferation.

There is a need for improved techniques and devices for assisting in healing of wounds in the GI tract, and particularly in the esophagus, by applying negative pressure to the vicinity of the wounds.

### SUMMARY

Various applications herein relate to medical systems and methods for removal of liquid from a target area in the GI tract, for example to assist in healing of an extraluminal or endoluminal wounds.

There is provided, in accordance with some applications, a medical system for applying negative pressure within a gastrointestinal tract of a subject. The medical system includes an elongate tube defining at least one interior channel along at least a longitudinal portion of the elongate tube, a plurality of orifices in fluid communication with the at least one interior channel, and at least one exterior channel extending longitudinally along an exterior surface of the elongate tube, at least one of the at least one exterior channel being in fluid communication with the at least one interior channel via at least one of the plurality of orifices. The elongate tube has a delivery state, and a first operative state in which the elongate tube forms a coil including a plurality of loops. The medical system further includes a fluid-tight lumen in fluid communication with an end of the elongate tube, the fluid-tight lumen adapted to couple to a source of negative pressure and to deliver negative pressure to the elongate tube. In the first operative state of the elongate tube, a length of the coil is at least 15 mm and the plurality of loops includes at least 4 loops.

In some embodiments, the at least one interior channel includes a first interior channel adapted for drainage of a fluid from the gastrointestinal tract, via the at least one exterior channel and the plurality of orifices, when negative pressure is applied to the elongate tube.

In some embodiments, the elongate tube includes a shape memory material, an elastic material, or a super-elastic material adapted to form the coil in the first operative state.

In some embodiments, the elongate tube has a textured exterior surface adapted to frictionally engage an interior surface of the gastrointestinal tract.

In some embodiments, the at least one exterior channel includes a plurality of exterior channels forming troughs on the exterior surface of the elongate tube and extending longitudinally therealong.

In some embodiments, the plurality of orifices are disposed about a single circumference of the elongate tube.

In some embodiments, the at least one exterior channel extends longitudinally along the exterior surface of the elongate tube between the orifices and a distal end of the elongate tube.

In some embodiments, the plurality of orifices are disposed longitudinally along a longitudinal length of the elongate tube.

In some embodiments, orifices in a first subset of the plurality of orifices have a first diameter, and orifices in a second subset of the plurality of orifices have a second diameter, the second diameter being different from the first diameter.

In some embodiments, in the first operative state, at least some of the plurality of orifices are oriented outwardly, away from a center of the coil.

In some embodiments, in the first operative state, at least some of the plurality of orifices are oriented inwardly, toward a center of the coil.

In some embodiments, in the first operative state of the elongate tube, at least one of the plurality of loops has a sinusoidal structure.

In some embodiments, the medical system further includes a linearizing element, wherein the elongate tube has the delivery state, which is a linear state, when associated with the linearizing element, and the first operative state, in which the elongate tube forms the coil, when dissociated from the linearizing element.

In some embodiments, the fluid-tight lumen, and the elongate tube in the delivery state, are sized and configured to pass through a working channel of a delivery device, wherein the working channel has a diameter smaller than 5 mm.

In some embodiments, the delivery state of the elongate tube is a coiled state, and the elongate tube is adapted to be delivered into the gastrointestinal tract of the subject disposed about the exterior of a delivery device.

There is provided, in accordance with some applications, a medical system for applying negative pressure within a gastrointestinal tract of a subject. The medical system includes an elongate tube defining at least one channel along at least a longitudinal portion thereof and including a plurality of orifices in fluid communication with the at least one channel the elongate tube having a delivery state and a first operative state in which the elongate tube forms a coil including a plurality of loops, at least some of the plurality of loops having a sinusoidal structure. The medical system further includes a fluid-tight lumen in fluid communication with an end of the elongate tube, the fluid-tight lumen adapted to couple to a source of negative pressure and to deliver negative pressure to the at least one channel via the end of the elongate tube. In the first operative state of the elongate tube, a length of the coil is at least 15 mm and the plurality of loops includes at least 4 loops.

In some embodiments, the at least one channel is adapted for drainage of a fluid from the gastrointestinal tract, via the plurality of orifices, when negative pressure is applied to the elongate tube.

In some embodiments, the elongate tube includes a shape memory material, an elastic material, or a super-elastic material adapted to form the coil in the first operative state.

In some embodiments, the elongate tube has a textured exterior surface adapted to frictionally engage an interior surface of the gastrointestinal tract.

In some embodiments, the plurality of orifices are disposed about a single circumference of the elongate tube.

In some embodiments, the plurality of orifices are disposed longitudinally along a longitudinal length of the elongate tube.

In some embodiments, orifices in a first subset of the plurality of orifices have a first diameter, and orifices in a second subset of the plurality of orifices have a second diameter, the second diameter being different from the first diameter.

In some embodiments, in the first operative state, at least some of the plurality of orifices are oriented outwardly, away from a center of the coil.

In some embodiments, in the first operative state, at least some of the plurality of orifices are oriented inwardly, toward a center of the coil.

In some embodiments, the medical system further includes a linearizing element wherein the elongate tube has the delivery state, which is a linear state, when associated with the linearizing element, and the first operative state, in which the elongate tube forms the coil, when dissociated from the linearizing element.

In some embodiments, the fluid-tight lumen, and the elongate tube in the delivery state, are sized and configured to pass through a working channel of a delivery device, wherein the working channel has a diameter smaller than 5 mm.

In some embodiments, the delivery state of the elongate tube is a coiled state, and the elongate tube is adapted to be delivered into the gastrointestinal tract of the subject disposed about the exterior of a delivery device.

There is provided, in accordance with some applications, a medical system for applying negative pressure within a gastrointestinal tract of a subject. The medical system includes an elongate tube defining at least one channel along at least a longitudinal portion thereof and including a plurality of orifices in fluid communication with the at least one channel. The elongate tube includes a coil portion forming a coil having a plurality of loops, and a linear portion extending through a longitudinal center of the coil portion. The medical system further includes a fluid-tight lumen in fluid communication with the elongate tube, the fluid-tight lumen adapted to couple to a source of negative pressure and to deliver negative pressure to the coil portion of the elongate tube. The elongate tube is adapted to be delivered into the gastrointestinal tract of the subject with the coil portion disposed about the exterior of a delivery device and the linear portion extending through the interior of the delivery device. A length of the coil is at least 15 mm and the plurality of loops includes at least 4 loops.

In some embodiments, the fluid tight lumen is in fluid communication with an end of the linear portion of the elongate tube, and is adapted to deliver the negative pressure to the coil portion of the elongate tube via the linear portion of the elongate tube.

In some embodiments, the at least one channel of the elongate tube includes at least one interior channel along at least a longitudinal portion of the elongate tube, and at least one exterior channel extending longitudinally along an exterior surface of the elongate tube, at least one of the at least one exterior channel being in fluid communication with the at least one interior channel via at least one of the plurality of orifices.

In some embodiments, the at least channel is adapted for drainage of a fluid from the gastrointestinal tract via the plurality of orifices, when negative pressure is applied to the elongate tube.

In some embodiments, at least the coil portion of the elongate tube has a textured exterior surface, adapted to frictionally engage an interior surface of the gastrointestinal tract.

In some embodiments, the plurality of orifices are disposed about a single circumference of the elongate tube.

In some embodiments, the plurality of orifices are disposed longitudinally along a longitudinal length of the elongate tube.

In some embodiments, orifices in a first subset of the plurality of orifices have a first diameter, and orifices in a second subset of the plurality of orifices have a second diameter, the second diameter being different from the first diameter.

In some embodiments, at least some of the plurality of orifices are oriented outwardly, away from a center of the coil.

In some embodiments, at least some of the plurality of orifices are oriented inwardly, toward a center of the coil.

In some embodiments, in the first operative state of the elongate tube, each of the plurality of loops has an at least partially sinusoidal structure.

There is provided, in accordance with some applications, a method of applying negative pressure to a portion of the gastrointestinal tract of a subject, the method including:
(a) placing within the gastrointestinal tract of the subject an elongate tube defining at least one channel along at least a longitudinal portion thereof and having a plurality of orifices in fluid communication with the at least one channel, the elongate tube forming a coil including a plurality of loops, an end of the elongate tube being in fluid communication with a first end of a fluid-tight lumen, wherein, within the gastrointestinal tract;
(b) coupling a second end of the fluid-tight lumen to a source of negative pressure; and
(c) while the elongate tube is in the form of the coil within the gastrointestinal tract, applying negative pressure to the gastrointestinal tract via the elongate tube and the fluid-tight lumen,
   wherein, within the gastrointestinal tract, a length of the coil is at least 15 mm and the plurality of loops includes at least four loops.

In some embodiments, the delivering includes delivering the elongate tube within a tubular sheath, and the forming includes advancing the elongate element distally out of the tubular sheath.

In some embodiments, the delivering includes advancing a linearizing wire into the elongate tube and delivering the elongate tube together with the linearizing wire, and the forming includes retracting the linearizing wire proximally relative to the elongate tube.

In some embodiments, the forming includes forming the plurality of loops sequentially, from the distal loop to the proximal loop.

In some embodiments, the method further includes delivering a fluid into the gastrointestinal tract via the fluid-tight lumen, the elongate tube, and the orifices.

There is provided, in accordance with some applications, a medical system for applying negative pressure within a gastrointestinal tract of a subject. The system includes an elongate tube defining at least one channel along at least a longitudinal portion thereof. The elongate tube includes a plurality of orifices, in fluid communication with the at least one channel. The elongate tube has a delivery state and a first operative state in which the elongate tube forms a coil including a plurality of loops. The medical system further includes a fluid-tight lumen in fluid communication with an end of the elongate tube. The fluid-tight lumen is adapted to couple to a source of negative pressure and to deliver negative pressure to the elongate tube, via the end of the elongate tube. In the first operative state of the elongate tube, a length of the coil is at least 15 mm or the plurality of loops includes at least 3 loops.

In some embodiments, the at least one channel extends longitudinally along an exterior surface of the elongate tube between the orifices and an end of the elongate tube.

In some embodiments, the at least one channel extends longitudinally along an exterior surface of the elongate tube, for example between the orifices and/or between the orifices an end of the elongate tube. In some embodiments, the end may be the distal end of the tube, and in some embodiments the end may be the proximal end of the tube.

In some embodiments, the elongate tube is formed of a porous material.

In some embodiments, the elongate tube has antimicrobial or anti-inflammatory properties. In some embodiments, the elongate tube includes an antimicrobial or anti-inflammatory material. In some embodiments, the elongate tube is pretreated or coated with at least one antimicrobial or anti-inflammatory agent.

In some embodiments, the gastrointestinal tract of the subject has an endoluminal wound, and the elongate tube is adapted to deliver to the vicinity of the endoluminal wound an antimicrobial or anti-inflammatory medicament for treatment of the endoluminal wound.

In some embodiments, the elongate tube has a textured exterior surface. In some embodiments, the textured exterior surface is adapted to frictionally engage an interior surface of the gastrointestinal tract.

In some embodiments, at least a portion of the elongate tube is detachable from the fluid-tight lumen. In some embodiments, the at least a portion of the elongate tube is formed from a biocompatible material and is adapted to be naturally excreted from the body of the subject following detachment from the fluid-tight lumen. In some other embodiments, the at least a portion of the elongate tube is formed from a biodegradable material, and is adapted to be degraded or decomposed, within the body of the subject, following detachment from the fluid-tight lumen.

In some embodiments, the at least one channel includes a first channel adapted for drainage of a fluid from the gastrointestinal tract, via the plurality of orifices, when negative pressure is applied to the elongate tube. In some embodiments, the first channel adapted for drainage of a fluid from the gastrointestinal tract, is further adapted to delivery fluid (e.g., flushing fluid containing a contrast material) to the gastrointestinal tract via the plurality of orifices.

In some embodiments, the at least one channel includes a second channel adapted for flushing a portion of the gastrointestinal tract, by supplying a flushing fluid into the gastrointestinal tract via the elongate tube and via at least some of the orifices.

In some embodiments, the fluid-tight lumen and the elongate tube are substantially concentric.

In some embodiments, the elongate tube includes a shape memory material, an elastic material, or a super-elastic material adapted to form the coil in the first operative state. Examples of the elastic material, super elastic material or shape memory material include for example a shape memory alloy, a spring alloy, a polymer or nitinol. In some embodiments, the elongate tube consists essentially of an elastic material, super elastic material or shape memory material.

In some embodiments, the medical system further includes a wire extending through or embedded within the elongate tube, the wire being an elastic wire, a super-elastic wire, or a shape memory wire. In some embodiments, the at least one channel includes a wire-accommodating channel adapted to accommodate the wire. In some other embodiments, the elongate tube has the wire embedded therein.

In some embodiments, the wire has a width not greater than 0.7 mm. A width in this context may be the second longest dimension of a cross section, in a direction perpendicular to a longitudinal axis of the wire.

In some embodiments, the wire is tubular. In some embodiments, the wire is a flat wire. In some embodiments, the wire has a circular, oval, I-shaped, C-shaped or D-shaped cross section, in a direction perpendicular to a longitudinal axis of the wire. In some embodiments, the wire has a polygonal cross section, in a direction perpendicular to a longitudinal axis of the wire.

In some embodiments, at least one end of the wire is protected by a blunt cover.

In some embodiments, the elongate tube further includes a plurality of channels forming troughs on an exterior surface of the elongate tube and extending longitudinally therealong, each of the plurality of troughs being in fluid communication with the at least one channel via at least one of the orifices. In some embodiments, negative pressure delivered to the orifices via the fluid-tight lumen results in negative pressure in the plurality of troughs draining fluid from the plurality of troughs, via the orifices, into the at least one channel of the elongate tube.

In some embodiments, the plurality of orifices are disposed about a single circumference of the elongate tube.

In some embodiments, the plurality of orifices are disposed longitudinally along a longitudinal length of the elongate tube.

In some embodiments, the plurality of orifices are equidistantly distributed along or about the elongate tube. In some other embodiments, the orifices are heterogeneously distributed along or about the elongate tube.

In some embodiments, each of the plurality of orifices has substantially the same diameter. In some other embodiments, orifices in a first subset of said plurality of orifices have a first diameter, and orifices in a second subset of said plurality of orifices have a second diameter, the second diameter being different from the first diameter.

In some embodiments, in the first operative state, at least some of the plurality of orifices are oriented inwardly, toward a center of the coil. In some embodiments, in the first operative state, at least some of the plurality of orifices are oriented outwardly, away from a center of the coil.

In some embodiments, in the first operative state, the coil is substantially devoid of orifices oriented outwardly, away from a center of the coil.

In some embodiments, the plurality of loops includes at least two loops having substantially the same diameter. In some embodiments, the at least two loops include a proximal-most loop and a distal-most loop of the plurality of loops.

In some embodiments, the diameter of one of the plurality of loops other than the proximal-most loop and the distal-most loop is not greater than the diameter of the proximal-most loop. In some embodiments, the diameter of one of the plurality of loops other than the proximal-most loop and the distal-most loop is not smaller than the diameter of the proximal-most loop.

In some embodiments, the at least two loops having substantially the same diameter includes a first loop and a second loop, and wherein a distance between the first loop and the second loop, along a longitudinal axis of the coil, is greater than 10 mm.

In some embodiments, in the first operative state of the elongate tube, the plurality of loops includes at least 4 loops. In some embodiments, in the first operative state of the elongate tube, the plurality of loops includes at least 5 loops. In some embodiments, in the first operative state of the elongate tube, the plurality of loops includes at least 8 loops. In some embodiments, in the first operative state of the elongate tube, the plurality of loops includes at least 10 loops.

In some embodiments, in the first operative state of the elongate tube, a number of loops in the plurality of loops is within a range of 4 to 25. In some embodiments, in the first operative state of the elongate tube, a number of loops in the plurality of loops is within a range of 5 to 25. In some embodiments, in the first operative state of the elongate tube, a number of loops in the plurality of loops is within a range of 4 to 15. In some embodiments, in the first operative state of the elongate tube, a number of loops in the plurality of loops is within a range of 5 to 15. In some embodiments, in the first operative state of the elongate tube, a number of loops in the plurality of loops is within a range of 5 to 12. In some embodiments, in the first operative state of the elongate tube, a number of loops in the plurality of loops is within a range of 8 to 12.

In some embodiments, in the first operative state of the elongate tube, a diameter of the coil is in a range of 0.5 cm to 5 cm. In some embodiments, in the first operative state of the elongate tube, a diameter of the coil is in a range of 1 cm to 5 cm. In some embodiments, in the first operative state of the elongate tube, a diameter of the coil is in a range of 0.5 cm to 4 cm. In some embodiments, in the first operative state of the elongate tube, a diameter of coil is in a range of 1 cm to 4 cm. In some embodiments, in the first operative state of the elongate tube, a diameter of the coil is in a range of 2.0 cm to 4.0 cm. In some embodiments, in the first operative state of the elongate tube, a diameter of the coil is in a range of 0.5 cm to 3 cm. In some embodiments, in the first operative state of the elongate tube, a diameter of the coil is in a range of 1 cm to 3 cm. In some embodiments, in the first operative state of the elongate tube, a diameter of the coil is in a range of 0.5 cm to 3 cm. In some embodiments, in the first operative state of the elongate tube, a diameter of coil is in a range of 1 cm to 3 cm. In some embodiments, in the first operative state of the elongate tube, a diameter of the coil is in a range of 2 cm to 3 cm. In some embodiments, in the first operative state of the elongate tube, a diameter of the coil is in a range of 2.5 cm to 3.5 cm.

In some embodiments, in the first operative state of the elongate tube, a diameter of the coil is not greater than 1.5 cm.

In some embodiments, in the first operative state of the elongate tube, each of the plurality of loops has an at least partially sinusoidal structure. In some embodiments, a space is created between each adjacent pair of loops of the plurality of loops.

In some embodiments, in the first operative state of the elongate tube, at least a subset of the plurality of loops are adapted to apply pressure, in a radial direction, to an interior surface of the gastrointestinal tract. In some embodiments, in the second operative state of the elongate tube, at least a subset of the plurality of loops are adapted to apply pressure, in a radial direction, to an interior surface of the gastrointestinal tract. In some embodiments, the pressure in the radial direction applied to the interior surface of the gastrointestinal tract by the subset of the plurality of loops is sufficient to retain the coil stationary within the gastrointestinal tract, for example for a duration of at least 6 hours.

In some embodiments, when the elongate tube is in the first operative state and negative pressure is applied to the elongate tube, the plurality of loops form a stack.

In some embodiments, when the elongate tube is in the first operative state and negative pressure is applied to the elongate tube, the plurality of loops tilt relative to a longitudinal axis of the coil.

In some embodiments, in the first operative state of the elongate tube, a length of the coil is at least 15 mm. In some embodiments, in the first operative state of the elongate tube, a length of the coil is at least 20 mm.

In some embodiments, in the first operative state of the elongate tube, a length of the coil is at most 200 mm. In some embodiments, in the first operative state of the elongate tube, a length of the coil is at most 150 mm. In some embodiments, in the first operative state of the elongate tube, a length of the coil is at most 100 mm. In some embodiments, in the first operative state of the elongate tube, a length of the coil is at most 80 mm. In some embodiments, in the first operative state of the elongate tube, a length of the coil is at most 70 mm. In some embodiments, in the first operative state of the elongate tube, a length of the coil is at most 60 mm. In some embodiments, in the first operative state of the elongate tube, a length of the coil is at most 50 mm.

In some embodiments, when a negative pressure, for example of 50-350 mmHg, is applied to the elongate tube, a second length of the coil is in the range of 10 mm to 50 mm. In some embodiments, when a negative pressure, for example of 50-350 mmHg, is applied to the elongate tube, a second length of the coil is in the range of 10 mm to 40 mm. In some embodiments, when a negative pressure, for example of 50-350 mmHg, is applied to the elongate tube, a second length of the coil is in the range of 20 mm to 50 mm. In some embodiments, when a negative pressure, for example of 50-350 mmHg, is applied to the elongate tube, a second length of the coil is in the range of 20 mm to 40 mm.

In some embodiments, in the first operative state of the elongate tube, the coil has a first longitudinal length, measured in ex-vivo pig esophagus, when negative pressure, for example in the range of 50-350 mmHg, is applied to the elongate tube, the coil has a second longitudinal length, measured in the ex-vivo pig esophagus, and a ratio between the first longitudinal length and the second longitudinal length is in the range of 1:1 to 4:1. In some embodiments, the ratio is in the range of 1:1 to 3:1. In some embodiments, the ratio is in the range of 1:1 to 2:1. In some embodiments, the ratio is in the range of 1:1 to 1.5:1. In some embodiments, the ratio is in the range of 1:1 to 1.25:1.

In some embodiments, a difference between the first longitudinal length and the second longitudinal length is not greater than 150 mm. In some embodiments, the difference is not greater than 125 mm. In some embodiments, the difference is not greater than 100 mm. In some embodiments, the difference is not greater than 75 mm. In some embodiments, the difference is not greater than 50 mm. In some embodiments, the difference is not greater than 40 mm. In some embodiments, the difference is not greater than 30 mm. In some embodiments, the difference is not greater than 20 mm. In some embodiments, the difference is not greater than 10 mm.

In some embodiments, in the first operative state of the elongate tube, the coil has a first cross-sectional area, measured in ex-vivo pig esophagus, when negative pressure, for example in the range of 50-350 mmHg, is applied to the elongate tube, the coil has a second cross-sectional area, measured in the ex-vivo pig esophagus, and a difference between a longest dimension of the first cross-sectional area and the second cross sectional area is less than 75% as measured in the ex-vivo pig esophagus. In some embodiments, the difference is less than 70%. In some embodiments, the difference is less than 60%. In some embodiments, the difference is less than 50%. In some embodiments, the difference is less than 40%. In some embodiments, the difference is less than 30%. In some embodiments, the difference is less than 20%.

In some embodiments, the first cross-sectional area is angled at a first angle relative to a longitudinal axis of the coil, the coil has a second cross-sectional area is angled at a second angle relative to the longitudinal axis of the coil, and the second angle is greater than the first angle.

In some embodiments, in the first operative state, a pitch of the coil is in a range of 2.5 mm to 25 mm. In some embodiments, in the first operative state, a pitch of the coil is in a range of 5 mm to 25 mm. In some embodiments, in the first operative state, a pitch of the coil is in a range of 2 mm to 40 mm. In some embodiments, in the first operative state, a pitch of the coil is in a range of 2 mm to 30 mm. In some embodiments, in the first operative state, a pitch of the coil is in a range of 2 mm to 25 mm. In some embodiments, in the first operative state, a pitch of the coil is in a range of 2 mm to 20 mm. In some embodiments, in the first operative state, a pitch of the coil is in a range of 3 mm to 15 mm.

In some embodiments, when the elongate tube is in the first operative state and negative pressure, for example in the range of 50-350 mmHg, is applied to the elongate tube, a second pitch of the coil is in the range of 0.2 cm to 2.5 cm.

In some embodiments, a ratio between the pitch and the second pitch is in the range of 1:1-4:1.

In some embodiments, when the elongate tube is in the first operative state, a pitch of the coil facilitates contraction and expansion of the coil in response to contraction and expansion of the gastrointestinal tract, such that a longitudinal position of the coil within the gastrointestinal tract, is retained during motion of the gastrointestinal tract.

In some embodiments, at least one characteristic of the coil is configurable by making a change to a condition in an environment surrounding the coil. In some embodiments, the at least one characteristic includes a chemical characteristic or a mechanical characteristic of the coil. In some embodiments, the condition includes a temperature.

In some embodiments, the medical system further includes a linearizing element, wherein the elongate tube is in the delivery state when associated with the linearizing element and obtains the first operative state when dissociated from the linearizing element. In some embodiments, the linearizing element is adapted to be removed from the gastrointestinal tract of the subject following delivery of the elongate tube.

In some embodiments, in the delivery state, the elongate tube is substantially linear.

In some embodiments, the linearizing element is a tubular sheath defining a lumen, and wherein the elongate tube is adapted to be delivered into the gastrointestinal tract within the lumen of the tubular sheath. In some embodiments, the elongate tube is adapted to be removed from the gastrointestinal tract via the lumen of the tubular sheath, and to assume the delivery state during the removal.

In some embodiments, the elongate tube is adapted to form the coil sequentially as segments of the elongate tube are pushed distally out of the tubular sheath, from the distal loop to the proximal loop.

In some embodiments, during removal of the elongate tube from the gastrointestinal tract, each of the loops of the coil is adapted to transform into a substantially linear segment, sequentially, from the proximal loop to the distal loop.

In some embodiments, a distal end of the tubular sheath includes a pointed shape.

In some embodiments, the linearizing element is a first wire, and the elongate tube is adapted to be delivered into the gastrointestinal tract with the first wire extended internally therethrough. In some embodiments, the first wire is a guidewire. In some embodiments, the medical system further includes a second wire, the second wire being a guidewire.

In some embodiments, the elongate tube includes a first channel in fluid communication with the fluid-tight lumen and a second channel, for example adapted for passage of the wire or the guidewire therethrough.

In some embodiments, the elongate tube is adapted to form the coil sequentially, as the wire is retracted proximally out of the elongate tube, from the distal loop to the proximal loop.

In some embodiments, the linearizing element terminates in a shoulder at an end thereof, the shoulder adapted to control a direction of the longitudinal axis of the coil.

In some embodiments, the medical system further includes a handle portion mechanically couplable to a second end of the fluid tight lumen, far from the elongate tube, such that manipulation of the handle portion results in distal motion of the elongate tube. In some embodiments, the manipulation includes pushing or turning of the handle portion.

In some embodiments, the handle portion is adapted to be detached from the fluid tight lumen following delivery of the elongate tube into the gastrointestinal tract.

In some embodiments, the manipulation of the handle portion causes the elongate tube to transition from the delivery state to the first operative state. In some embodiments, the transition is sequential, such that each of the plurality of loops is adapted to form as the elongate tube is delivered into the gastrointestinal tract, sequentially from the distal loop to the proximal loop.

In some embodiments, the fluid-tight lumen, and the elongate tube in the delivery state, are sized and configured to pass through a working channel of a delivery device. In some embodiments, a working channel has a largest cross-sectional diameter of less than 5 mm. In some embodiments, a working channel has a largest cross-sectional diameter of 4 mm. In some embodiments, a working channel has a largest cross-sectional diameter of 3.5 mm. In some embodiments, an external delivery sheath, adapted to be disposed above the elongate tube during delivery thereof, is sized and configured to pass through the working channel of the delivery device.

In some embodiments, the elongate tube and the fluid tight lumen are adapted to be mechanically attached to an exterior of a delivery device during delivery into the gastrointestinal tract, and to be detached from the delivery device when the elongate tube is positioned within the gastrointestinal tract. In some embodiments, in the delivery state, the elongate tube is coiled about the exterior of the delivery device.

In some embodiments, the delivery device includes a catheter or an endoscope.

In some embodiments, the delivery device further includes an image capturing element, adapted to provide images of the elongate tube during delivery thereof into the gastrointestinal tract.

In some embodiments, the medical system further includes the delivery device.

In some embodiments, the medical system further includes the source of negative pressure functionally associated with the fluid-tight lumen, the source of negative pressure being adapted to apply a negative pressure to the elongate tube, for example in the range of 50-350 mmHg.

In some embodiments, the source of negative pressure includes a controller adapted to regulate the negative pressure provided by the source of negative pressure, within a pressure range, to remove fluid from the vicinity of at least a portion of the internal surface of the gastrointestinal tract.

In some embodiments, the elongate tube is adapted to deliver negative pressure to a portion of the gastrointestinal tract including an internal wound including, for example, a fistula, and wherein the controller is adapted to regulate the negative pressure for removal of fluid from the vicinity of an internal surface of the body conduit including the internal wound including for example, a fistula.

In some embodiments, the medical system further includes at least one sensor adapted to sense at least one characteristic of the fluid removed from the vicinity of the at least a portion of the gastrointestinal tract, and wherein the controller is adapted to adjust one or more operating parameters of the source of negative pressure in response to input received from the at least one sensor, the information relating to the at least one characteristic of the fluid.

In some embodiments, the medical system further includes a fluid source functionally associated with the fluid-tight lumen, and adapted to provide fluid to the elongate tube, via the fluid tight lumen, for flushing the vicinity of the orifices.

In some embodiments, the medical system further includes an internal support tube, adapted to be placed within an internal cavity of the coil and to support the coil from within. In some embodiments, the internal support tube has atmospheric pressure when the elongate tube is in the first and in the second operative states.

There is provided, in accordance with some applications, a method of applying negative pressure to a portion of the gastrointestinal tract of a subject, the method including: (a) placing within the gastrointestinal tract of the subject an elongate tube defining at least one channel along at least a longitudinal portion thereof and having a plurality of orifices in fluid communication with the at least one channel, the elongate tube forming a coil including a plurality of loops, an end of the elongate tube being in fluid communication with a first end of a fluid-tight lumen, wherein, within the gastrointestinal tract; (b) coupling a second end of the fluid-tight lumen to a source of negative pressure; and (c) while the elongate tube is in the form of the coil within the gastrointestinal tract, applying negative pressure to the gastrointestinal tract via the elongate tube and the fluid-tight lumen.

In some embodiments, a length of the coil is at least 15 mm or the plurality of loops includes at least four loops.

In some embodiments, placing includes delivering the elongate tube into the gastrointestinal tract of the subject in a delivery state.

In some embodiments, delivering includes delivering the elongate tube when the elongate tube is substantially linear, and wherein the placing further includes forming the coil within the gastrointestinal tract of the subject.

In some embodiments, the elongate tube includes a shape memory material, delivering includes delivering the elongate tube in association with a linearizing element, the linearizing element maintaining the substantially linear state of the elongate tube during the delivery, and forming includes dissociating the linearizing element from the elongate tube.

In some embodiments, delivering includes delivering the elongate tube within a tubular sheath, and the forming includes advancing the elongate element distally out of the tubular sheath.

In some embodiments, delivering includes advancing a linearizing wire into the elongate tube and delivering the elongate tube together with the linearizing wire, and the forming includes retracting the linearizing wire proximally relative to the elongate tube.

In some embodiments, forming includes forming the plurality of loops sequentially, from the distal loop to the proximal loop.

In some embodiments, delivering and forming include manipulating a handle portion mechanically coupled to the second end of the fluid tight lumen, which manipulation of the handle portion results in distal motion of the elongate tube. In some embodiments, manipulating includes pushing or turning of the handle portion.

In some embodiments, the method further includes detaching the handle portion from the fluid tight lumen following the delivering and prior to the coupling of the fluid-tight lumen to the source of negative pressure.

In some embodiments, manipulating of the handle portion causes the elongate tube to transition from a linear configuration into the coil.

In some embodiments, delivering includes advancing the elongate tube through a working channel of a delivery device.

In some embodiments, placing includes mechanically attaching the elongate tube to an exterior of a delivery device, advancing the delivery device, together with the elongate tube, into the lumen of the human body, and when the elongate tube is positioned in the vicinity of the wound (for example, a fistula) within the lumen, detaching the elongate tube from the delivery device. In some embodiments, mechanically attaching includes forming the coil about the exterior of the delivery device, the method further including retracting the delivery device from the gastrointestinal tract, thereby to leave the coil placed in the gastrointestinal tract.

In some embodiments, during the delivering, the elongate tube is mechanically associated with an imaging device, the method further including imaging the elongate tube during advancing of the elongate tube into the gastrointestinal tract.

In some embodiments, applying of the negative pressure includes applying negative pressure in the range of 50-350 mmHg.

In some embodiments, applying negative pressure includes using a controller, regulating the negative pressure applied to the portion of the gastrointestinal tract for removal of fluid from the portion of the gastrointestinal tract.

In some embodiments, the method further includes receiving from at least one sensor input relating to at least one characteristic of the fluid removed from the gastrointestinal tract and in response to the received input, using the controller, adjusting one or more operating parameters of the source of negative pressure.

In some embodiments, the portion of the gastrointestinal tract includes an endoluminal wound for example a fistula in the gastrointestinal tract, and the applying negative pressure removes fluid from the vicinity of the endoluminal wound for example a fistula.

In some embodiments, the method further includes following the applying of the negative pressure, removing the elongate tube from the gastrointestinal tract. In some embodiments, removing includes pulling the second end of the fluid tight lumen in a proximal direction. In some embodiments, removing includes transforming the elongate tube from the coil into a substantially linear state, such that each loop forms a substantially linear segment, from the proximal loop to the distal loop.

In some embodiments, the method further includes flushing a flushing-fluid into the gastrointestinal tract via the fluid-tight lumen, the elongate tube, and the orifices.

In some embodiments, the method further includes, prior to the flushing, connecting a fluid source containing the flushing-fluid to the second end of the fluid tight lumen.

In some embodiments, the method further includes, prior to the placing, forming the elongate tube out of a shape memory material.

In some embodiments, the method further includes, prior to the placing, embedding in the elongate tube at least one shape-memory wire.

In some embodiments, the method further includes, prior to the delivering, threading through a channel in the elongate tube a shape-memory wire.

In some embodiments, the method further includes, prior to the placing of the elongate tube, pretreating the elongate tube with at least one antimicrobial or anti-inflammatory agent.

In some embodiments, the method further includes delivering to the portion of the gastrointestinal tract, via the elongate tube, an antimicrobial or anti-inflammatory medicament for treatment of an endoluminal wound in the portion of the gastrointestinal tract.

In some embodiments, the method further includes, following the applying of the negative pressure and while the elongate tube is within the gastrointestinal tract, detaching at least a portion of the elongate tube from the fluid-tight lumen, wherein (a) the at least a portion is formed from a biocompatible material and is naturally excreted from the body of the subject following the detaching and/or (b) the at least a portion is formed from a biodegradable material, and is degraded or decomposed, within the body of the subject, following the detaching.

In some embodiments, the method further includes changing a condition in an environment surrounding the coil, thereby configuring at least one characteristic of the coil. In some embodiments, changing includes changing a temperature in the vicinity of the coil.

### BRIEF DESCRIPTION OF THE FIGURES

The foregoing discussion will be understood more readily from the following detailed description when taken in conjunction with the accompanying Figures, in which:

FIG. 1 is a schematic illustration of a medical system according to embodiments of the disclosed technology, deployed in the esophagus;

FIGS. 2A, 2B, and 2C are sectional illustrations of various exemplary structures of an elongate tube forming part of the medical system of FIG. 1, according to embodiments of the disclosed technology;

FIG. 2D and 2E are perspective sectional illustration of an exemplary structure of an elongate tube forming part of the medical system of FIG. 1, according to embodiments of the disclosed technology;

FIGS. 3A, 3B, and 3C are, respectively, side view illustrations and an end view illustration of an exemplary structure of an elongate tube forming part of the medical system of FIG. 1, according to embodiments of the disclosed technology;

FIGS. 4A, 4B, and 4C are schematic illustrations of exemplary structures of a coil formed of an elongate tube forming part of the medical system of FIG. 1, according to embodiments of the disclosed technology;

FIGS. 5A, 5B and 5C are schematic illustrations of steps of deploying the medical system of FIG. 1 into the gastrointestinal tract according to an embodiment of the disclosed technology;

FIGS. 6A and 6B are schematic illustrations of steps of deploying the medical system of FIG. 1 into the gastrointestinal tract according to an embodiment of the disclosed technology;

FIGS. 7A and 7B are schematic illustrations of steps of deploying the medical system of FIG. 1 into the gastrointestinal tract according to an embodiment of the disclosed technology;

FIGS. 8A and 8B are schematic illustrations of steps of deploying the medical system of FIG. 1 into the gastrointestinal tract according to an embodiment of the disclosed technology;

FIGS. 9A and 9B are schematic illustrations of a method of deploying the medical system of FIG. 1 into the gastrointestinal tract, using a delivery device, according to an embodiment of the disclosed technology;

FIGS. 10A and 10B are schematic illustrations of a procedure of deploying a medical system, similar to that of FIG. 1, into the body of a subject, according to embodiments of the disclosed technology; and

FIGS. 11A, 11B, 11C, 11D, and 11E are schematic illustrations of steps of a procedure for maintaining the medical system of FIG. 1 in the body of the subject via a nasal wire or tube.

### DETAILED DESCRIPTION

The principles of the medical systems and methods may be better understood with reference to the drawings and the following description.

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features can be omitted or simplified in order not to obscure the disclosure. Additionally, in order to avoid undue clutter from having too many reference numbers and lead lines on a particular drawing, some elements may not be explicitly identified in every drawing that contains that element.

It is to be understood that the scope of the invention is not limited in its application to the details of construction and the arrangement of the components set forth in the following description or illustrated in the drawings. The invention is capable of other implementations or of being practiced or carried out in various ways. Furthermore, it is to be understood that the phraseology and terminology employed in the disclosure is for the purpose of description and should not be regarded as limiting.

For the purposes of this application, the term “subject” relates to any mammal, particularly humans.

In the context of the present description and claims, the terms “proximal” and “distal” are defined relative to a direction in which the system is deployed into the body of the subject. As such, an element is said to be “proximal” if it is closer to the point at which the system enters the body of the subject than other elements, and is said to be “distal” if it is further from the point at which the system enters the body of the subject than other elements.

In the context of the present description and claims, the term “wound” relates to any form of damage to the tissue, including, but not limited to, a leak, a perforation, a rupture, a tear, a cut, or a fistula in the tissue, for example in the wall of the GI tract.

In the context of the present description and claims, the term “negative pressure” relates to sub-atmospheric pressure, which may be applied, for example, to remove fluid or debris from a bodily lumen.

In the context of the present description and claims, the term “elongate tube” relates to an elongate structure having at least a portion which is tubular, i.e. at least a portion that includes an internal channel. The internal channel need not extend through the entire elongate structure for the structure to be considered an “elongate tube”.

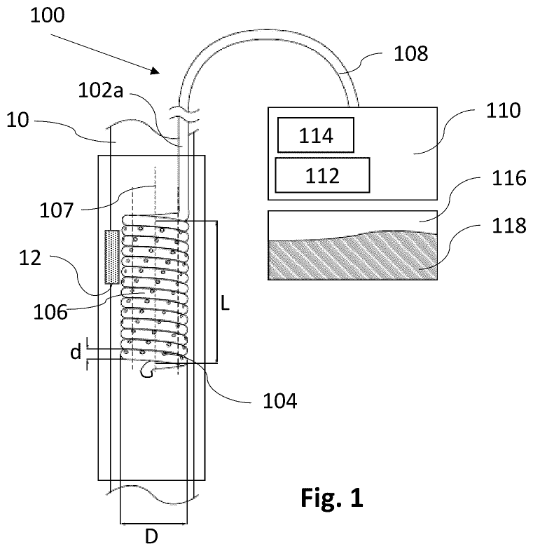

Referring now to the drawings, FIG. 1 is a schematic illustration of a medical system 100 according to embodiments of the disclosed technology, deployed in the vicinity of an endoluminal or extraluminal wound, for example in an esophagus 10 of a subject. Typically, system 100 is configured to remove fluid from the vicinity of wound 12, and/or to assist in healing thereof.

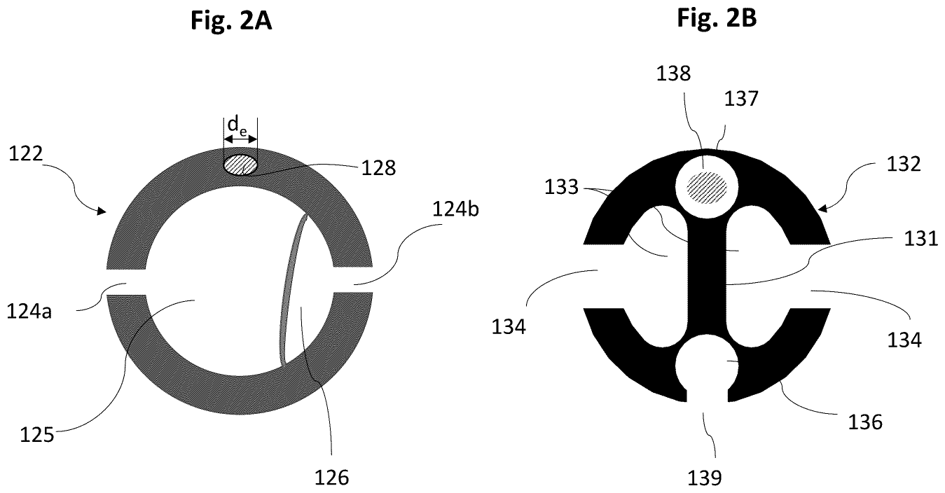

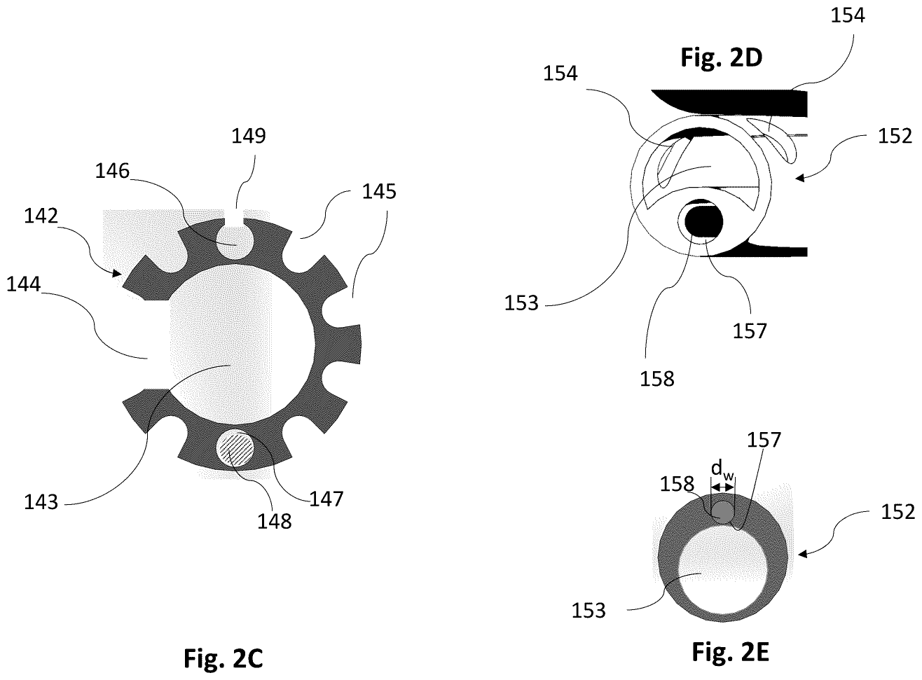

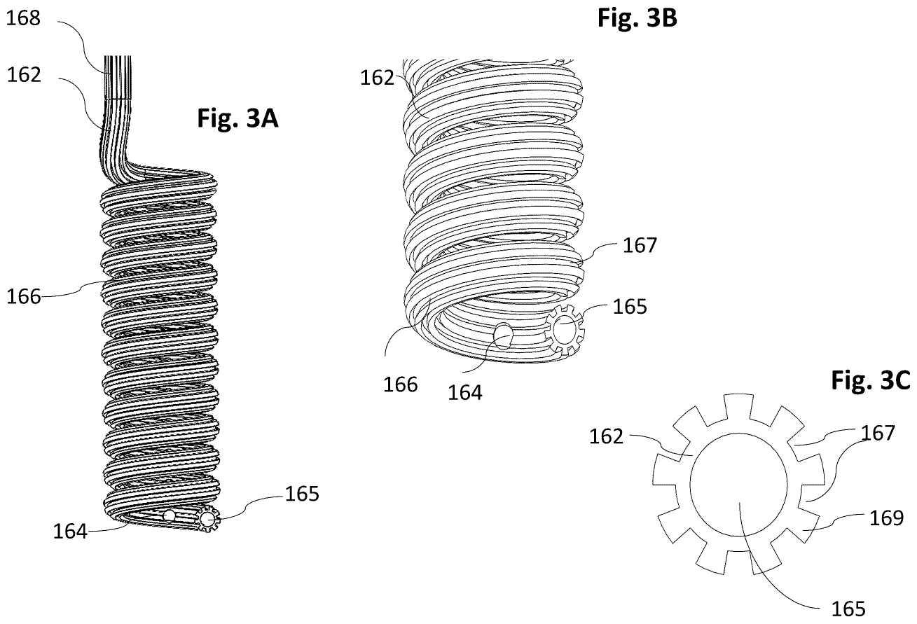

System 100 includes an elongate tube 102 defining at least one channel (see for example interior channel 125 in FIG. 2A) along at least a longitudinal portion thereof. In some embodiments, the at least one channel may extend along the entire longitudinal length of the elongate tube. In other embodiments, the at least one channel may extend only within a proximal portion of the elongate tube. The elongate tube 102 is shaped and sized for delivery to a human esophagus, or to another portion of the human GI tract. The elongate tube 102 includes a plurality of orifices 104, in fluid communication with channel(s). In some embodiments, such as that shown in FIG. 1, the orifices 104 may be distributed longitudinally along at least a portion of elongate tube 102. In other embodiments, such as that shown in FIG. 3A, the orifices may be distributed about a single circumference of elongate tube 102.

Elongate tube 102 has a delivery state, and a first operative state, also termed a resting operative state, as seen in FIG. 1, for example. The resting operative state occurs when the elongate tube is deployed in the body of the subject. In the resting state, the elongate tube forms a coil including a plurality of loops 106, arranged around a longitudinal coil axis 107. In the following description, the terms “first operative state” and “resting operative state” are used interchangeably.

A fluid-tight lumen 108 is in fluid communication with an end of elongate tube 102, typically with the channel(s) thereof. Fluid-tight lumen 108 is adapted to couple, or couples, elongate tube 102 to a source of negative pressure (e.g., negative pressure system) 110, for delivery of negative pressure to orifices 104 via elongate tube 102 and its channel(s). Negative pressure delivered to the orifices results in removal or drainage of fluid and/or debris from the vicinity of wound 12, thus assisting in healing of the wound.

In some embodiments, and as illustrated in FIG. 1, fluid-tight lumen 108 is in fluid communication with a proximal end 102a of elongate tube 102. Additionally, the fluid-tight lumen 108 is adapted to couple to a source of negative pressure 110, and to deliver negative pressure to the elongate tube 102 via the proximal end 102a. However, depending on the direction of deployment, fluid-tight lumen 108 may alternately be coupled to a distal end of the elongate tube, provided that it is coupled to source of negative pressure 110.

In some embodiments, the system includes the source of negative pressure 110, for example in the form of a vacuum generator, which may be fixed or portable. In other embodiments, the system does not include the source of negative pressure, and merely interacts or is adapted to connect with the source of negative pressure such as a vacuum system.

In some embodiments, source of negative pressure 110 includes a controller 112 adapted to regulate the negative pressure provided by source 110, within a predefined pressure range, to remove fluid at least from a vicinity of the extraluminal or endoluminal wound, a portion of the internal surface of esophagus 10, or of the gastrointestinal tract. For example, controller 112 may be adapted to regulate the negative pressure for removal of fluid from an area of esophagus 10 including the extraluminal or endoluminal wound.

In some embodiments, source of negative pressure 110 may further include, or be associated with, least one sensor 114 adapted to sense at least one characteristic of the fluid removed from the gastrointestinal tract such as pressure or flow rate. Sensor(s) 114 is functionally associated with controller 112, such that the controller is adapted to adjust one or more operating parameters of the source of negative pressure 110 in response to input received from the sensor(s), which input relates to the at least one characteristic of the fluid. Sensor(s) 114 may be positioned in proximity to controller 112, or in proximity to elongate tube 102.

In some embodiments, the channel spans a longitudinal portion of elongate tube 102 between orifices 104 and the proximal end of the elongate tube, coupled to fluid-tight lumen 108, but may not span the entire longitudinal length of the elongate tube. In some embodiments, the channel spans the entire length of elongate tube 102. In some embodiments, the channel is an inner channel, such as channel 125 shown in FIG. 3A.

In some embodiments, elongate tube 102 and fluid-tight lumen 108 are substantially concentric, such that elongate axes thereof are substantially coincidental. However, the coil axis 107 is typically not coincidental with the elongate axis of fluid-tight lumen 108.

In some embodiments, or at certain times, elongate tube 102 may be dissociated from the source of negative pressure 110, and may be coupled instead to a source of fluid, indicated by reference numeral 116 in FIG. 1. Source 116 includes fluid 118, which may be a flushing fluid or a treatment fluid. In some embodiments, fluid 118 may be supplied, via the channel(s) and orifices 104, into the gastrointestinal tract. For example, the fluid may be supplied to dislodge debris caught in the orifices or in the channel. As another example, the fluid may include an irrigation or cleansing fluid, a medicament (e.g., anti-inflammatory agent), and/or an antimicrobial (e.g., antibiotic or antibacterial), to assist in healing of wound 12.

In some embodiments, the fluid may be a flushing fluid. In some embodiments, the fluid may be a medicament fluid, such as an antimicrobial fluid or a tissue-growth promoting fluid. In some embodiments, the fluid may be a contrast fluid. In some embodiments, the fluid may be ionized gas. In some embodiments, the fluid may be carbon dioxide. In some embodiments, the fluid may be a fluid configured to modify a characteristic of the coil, such as a low temperature fluid.

In other embodiments, the source of fluid 116 as well as the source of negative pressure 110 may both be connected to elongate tube 102. For example, the at least one channel includes multiple channels, a first (e.g., 125 in FIG. 2A) connected to source of negative pressure 110 and another (e.g., 126 in FIG. 2A) connected to source of fluid 116, such that negative pressure and fluid may be provided to the vicinity of elongate tube 102, via different channels or orifices, substantially simultaneously, or at different times via different channels.

In some embodiments, upon completion of treatment, the entirety of elongate tube 102, as well as the fluid-tight lumen 108 are removed from the body of the subject.

In some embodiments, at least a portion of elongate tube 102, or the entirety of elongate tube 102, may be detachable from fluid-tight lumen 108. In some embodiments, the detachable portion (or entirety) of elongate tube 102 is formed of material which may be naturally excreted from the body of the subject following detachment from fluid tight lumen 108. In other embodiments, the detachable portion (or entirety) of elongate tube 102 may be formed of a biodegradable material, and may be degraded or decomposed, within the body of the subject, following detachment from fluid tight lumen 108.

In some embodiments, at least a portion of elongate tube 102, or the entirety thereof, is further covered by an additional layer of material such as a netting configured to add friction to the surface of the elongate tube.

In some embodiments, after the elongate tube 102 has formed the coil, an internal support tube 119 may be pushed into the center of the coil, to extend therethrough. In some embodiments, the internal support tube may be a feeding tube, adapted for delivery of food to the stomach of the subject, via the coil in the esophagus.

It is to be appreciated that the presence of the internal support tube 119 within the coil retains the shape of the coil and of the esophagus when negative pressure is applied to the coil, and ensures that the shape of the coil and the esophagus will not collapse. Furthermore, because the interior of the internal support tube 119 is at atmospheric pressure, the negative pressure region is limited to a smaller portion of the esophagus, thus improving the draining ability of the negative pressure delivered through the elongate tube, and the functionality of the coil.

In some embodiments, one or more of sensors 114 may be associated with the internal support tube 119, and may be adapted to sense, and provide an indication of, a change in pressure within internal support tube 119. In some such embodiments, upon identification of such a change in pressure, a fluid at atmospheric pressure (e.g. saline) may be delivered to the interior of the internal support tube, to ensure that it continues to function as desired, and does not collapse.

Reference is now additionally made to FIGS. 2A, 2B, 2C, 2D, and 2E which show exemplary structures of elongate tubes, each being similar to elongate tube 102 of FIG. 1, according to embodiments of the disclosed technology.

In FIG. 2A, an elongate tube 122 includes a plurality of orifices including a first subset of orifices 124a, and a second subset of orifices 124b. Elongate tube 122 defines a first, vacuum-delivery, channel 125 in fluid communication with a first subset of orifices 124a, and optionally a second, flushing, channel 126 in fluid communication with a second subset of orifices 124b.

In use of system 100, first channel 125 and the first subset of orifices 124a are in fluid communication with the source of negative pressure 110, such that fluid from the vicinity of the orifices in the first subset is drained, via those orifices and first channel 125. Second channel 126 and orifices 124b in the second subset are in fluid communication with source 116 of fluid, such that fluid supplied from source 116 flows through second channel 126 and orifices 124b into the vicinity of the orifices, such as into esophagus 10 in the vicinity of wound 12. The flushing fluid may be supplied continuous, intermittently, periodically or as needed.

In some embodiments, elongate tube 122 may comprise, or may consist of, a radiopaque marker, radioactive marker, magnetic marker, and/or magnetic resonance marker. In some embodiments, elongate tube 122 may comprise, or may consist of, a metal, a natural or elastic polymer, a plastic, a shape memory alloy, a super clastic alloy, a biodegradable material, a bioresorbable material, and/or a bioabsorbable material.

In some embodiments, elongate tube 122 may comprise, or may consist of, a shape memory, elastic or super-elastic material adapted to form the coil in the resting operative state. For example, in some embodiments, elongate tube 122 may be formed of the shape memory, clastic, or super-elastic material.

In some embodiments, elongate tube 122 may have embedded therein a wire or filament 128, configured to act as a frame for formation of a coil.

In the embodiment illustrated in FIG. 2B, an elongate tube 132, similar to tube 102 of FIG. 1, includes a central wall 131, defining a pair of channels 133. The elongate tube 132 includes a plurality of orifices 134 in fluid communication with channels 133, such that channels 133 form vacuum-delivery channels of elongate tube 132. Channels 133 are in fluid communication with source 110 of negative pressure, and function substantially as described hereinabove with respect to channel 125 of FIG. 2A.

A flushing channel 136 is formed in the circumferential wall of elongate tube 132. Typically, flushing channel 136 is associated with at least one orifice 139 in the wall of elongate tube 132. Flushing channel 136 is adapted to be in fluid communication with fluid source 116 for supply of flushing fluid 118 to the vicinity of the elongate tube, substantially as described hereinabove with respect to FIG. 2A.

A filament or wire-accommodating channel 137 is also formed in the wall of elongate tube 132, and is adapted to accommodate a wire or filament 138, adapted to form the coil in the resting operative state of elongate tube 132. Wire or filament 138 may be similar to wire 128, described hereinabove with respect to FIG. 2A.

In the embodiment illustrated in FIG. 2C, an elongate tube 142 which is similar to tube 102, includes a central channel 143 and orifices 144. An exterior surface of elongate tube 142 includes a plurality of troughs 145, extending longitudinally along the tube. As seen in FIG. 2C, in some embodiments, each of orifices 144 spans the width of multiple troughs 145. Stated differently, the diameter of troughs 145 is smaller than the diameter of orifices 144.

Channel 143 is in fluid communication with source 110 of negative pressure, and functions substantially as described hereinabove with respect to channel 125 of FIG. 2A. The negative pressure is applied to the vicinity of the tube 142 via orifices 144, and drains fluid and debris from the vicinity of the tube.

Troughs 145 fulfill multiple purposes in the treatment using the system of the disclosed technology. The presence of troughs 145 assists in maintaining orifices 144 open, particularly when coils 106 (FIG. 1) of the elongate tube are disposed directly one over the other, with no gaps. In such conditions, troughs 145 form a channel through which the negative pressure can be applied to the vicinity, even if the coils engage one another. Additionally, troughs 145, which have a narrow cross section, are delineated by ridges, which ridges can engage the surrounding tissue, such as tissue of esophagus 12, and promote tissue growth, thereby to accelerate healing. Furthermore, in some embodiments, fluid may be drained via troughs 145 into orifices 144, thus facilitating drainage from a larger area using fewer orifices, and the orifices are less likely to be blocked or occluded by debris.

In some embodiments, elongate tube 142 may optionally further include a flushing channel 146 associated with fluid delivery orifices 149 for delivery of fluid to the vicinity of the elongate tube 142. The flushing channel 146 may be formed in the circumferential wall of elongate tube 142, as described hereinabove with respect to FIG. 2C.

In some embodiments, elongate tube 142 may optionally further include a wire-accommodating channel 147, formed in the circumferential wall of elongate tube 142, substantially as described hereinabove with respect to FIG. 2C. Wire-accommodating channel 147 is adapted to accommodate a wire or microfilament 148, substantially as described hereinabove with respect to wire 128.

In the embodiment illustrated in FIG. 2D and 2E, an elongate tube 152 similar to tube 102 includes a main channel 153, and orifices 154. Channel 153, which can have a variety of cross-sectional shapes as demonstrated in FIGS. 2D and 2E, is in fluid communication with source 110 of negative pressure, and functions substantially as described hereinabove with respect to channel 125 of FIG. 2A. In some embodiments, the cross-sectional area of the orifices 154 increases along the length of the elongate tube, or of the coil, from the proximal end towards the distal end. In some such embodiments, the cross-sectional area of the distal-most orifice is at least 50% greater than the cross-sectional area of the proximal-most orifice.

Elongate tube 152 further includes a wire-accommodating channel 157, adapted to accommodate a wire 158. Wire 158 is adapted to form the coil in the resting operative state of elongate tube 152, as described hereinabove. As used herein, a wire 158 may be a wire or filament.

The structures illustrated in any one of FIGS. 2A-2E may have several advantages, which are described herein, for brevity, with respect to the structure and reference numerals of FIG. 2E.

In some embodiments, wire 158 may have a lower yield strain than elongate tube 152. Additionally, elongate tube 152 may be coextruded with wire 158, for example from two polymeric materials. For example, wire 158 may be formed of a material having a higher Young's modulus value than the material of the elongate tube 152. Such selection of materials is enabled by the yield strain on the wire being low. In some embodiments, both the elongate tube 152 and wire 158 are formed of thermoplastic materials having a thermoforming temperature to allow plastic deformation to form a coil shape for example in the range of 80-150 degrees Celsius, and a melting point above the thermoforming temperature. The elongate tube 152 and wire 158 has Young's modulus E that meets the following equation, where I is the second moment of inertia:

$${E}_{wire} * {I}_{wie} \geq \frac{{E}_{ElongatedTube} * {I}_{El ongatedTube}}{2}$$

In some embodiments, any one of wires 128, 138, 148, or 158 may comprise, or be formed of, a shape-memory material or a super elastic material. In some embodiments, any one of wires 128, 138, 148, or 158 may comprise, or may be formed of, a spring alloy, such as nitinol. In some embodiments, the material of any one of wires 128, 138, 148, or 158 has critical yield strain or elastic strain of more than 3% (0.03). In some embodiments, the material of any one of the wires has a Young's modulus of at least 50 Mpa.

Any one of wires 128, 138, 148, or 158 may have any suitable shape or cross section. For example, the wire may be tubular, may be a flat wire, or may have a circular or polygonal cross section in a direction perpendicular to a longitudinal axis of the wire.

In some embodiments, any one of wires 128, 138, 148, or 158 may a circular cross section having diameter d_{w} (shown in FIG. 2E), or a non-circular cross section having a greatest cross-sectional length dₑ (shown in FIG. 2A). In some embodiments, dimension d_{w} or dₑ is smaller than 1.5 mm, 0.8 mm or 0.7 mm. In some embodiments, dimension d_{w} or dₑ is greater than 0.4 mm or greater than 0.5 mm. In some embodiments, dimension d_{w} or dₑ is in the range of 0.4 mm to 1.5 mm, 0.4 mm to 0.8 mm or about 0.5 mm to 0.7 mm.

In some embodiments, an end of any one of wires 128, 138, 148, or 158 may be blunt, or may be protected by a blunt structure. For example, the end of the wire may be protected by a plastic or silicone outer end, and by a soft tube or soft wire, as a tail.

Reference is now additionally made to FIGS. 3A, 3B, and 3C, which are, respectively, side view illustrations and an end view illustration of an exemplary structure of an elongate tube forming part of the medical system of FIG. 1, according to embodiments of the disclosed technology.

FIGS. 3A to 3C show an elongate tube 162, which has a similar function to elongate tube of FIG. 1, but differs therefrom in several aspects. Like elongate tube 102, which includes orifices 104 and forms a coil including a plurality of loops 106, elongate tube 162 includes orifices 164 and forms a coil including a plurality of loops 166.

Elongate tube 162 includes an internal channel 165, and in addition includes one or more exterior channels 167, here shown as a plurality of troughs extending longitudinally along an exterior surface of the elongate tube. Orifices 164, which, in the embodiment of FIGS. 3A to 3C, are disposed about a single circumference of the elongate tube, adjacent the distal end of the elongate tube, are in fluid communication with interior channel 165 as well as with at least one of exterior channels 167, and facilitate fluid flow between the internal channel and at least one of the exterior channels. However, in some embodiments, orifices 164 may be longitudinally distributed along a portion of the elongate tube or along the entirety of the elongate tube, for example as shown in FIG. 1. In the illustrated embodiment, fluid-tight lumen 168 has a similar structure to elongate tube 162, and also includes exterior channels.

As seen in FIG. 3B, in some embodiments, each of orifices 164 spans the width of multiple troughs, or exterior channels, 167. Stated differently, the cross section or diameter of troughs 167 is smaller than the diameter of orifices 164.

Channel 165 is in fluid communication with source 110 of negative pressure, via fluid tight lumen 168, and functions substantially as described hereinabove with respect to FIG. 1. The negative pressure is applied to the vicinity of the elongate tube 162 via orifices 164 and troughs 167, and drains fluid and debris from the vicinity of the tube, via troughs 167 and orifices 164, into channel 165.

It is to be appreciated that in some embodiments, orifices 164 may be disposed about a single circumference of elongate tube 162, adjacent the proximal end of the elongate tube. In some such embodiments, internal channel 165 must extend along a proximal longitudinal portion of the elongate tube leading up to, or slightly past, orifices 164, but need not necessarily extend beyond orifices 164. In such embodiments, negative pressure would be delivered from fluid-tight lumen, via the portion of internal channel 165 and the orifices 164 to a proximal end of troughs 167, such that fluid and debris from the vicinity of the coil is drawn longitudinally along troughs 167 from the distal end toward the proximal end, and from there through orifices 164 into internal channel 165.

Troughs 167 fulfill multiple purposes in the treatment using the system of the disclosed technology. The presence of troughs 167 assists in maintaining orifices 164 open, particularly when loops 166 of the elongate tube are disposed directly one over the other, with no gaps. In such conditions, troughs 167 may form a channel through which the negative pressure can be applied to the vicinity, even if the coils engage one another. Additionally, troughs 167, which have a narrow cross section, are delineated by ridges 169. These ridges provide a texture to the exterior surface of elongate tube 162, and can engage the surrounding tissue, such as tissue of esophagus 12, to promote tissue growth, thereby to accelerate healing. Furthermore, in some embodiments, fluid may be drained via troughs 167 into orifices 164, thus facilitating drainage from a larger area using fewer orifices, and the orifices are less likely to be blocked or occluded by debris.

In some embodiments, elongate tube 162 may optionally further include a second channel associated with fluid delivery orifices (not explicitly shown) for delivery of fluid to the vicinity of the elongate tube 162, substantially as described hereinabove with respect to FIG. 1.

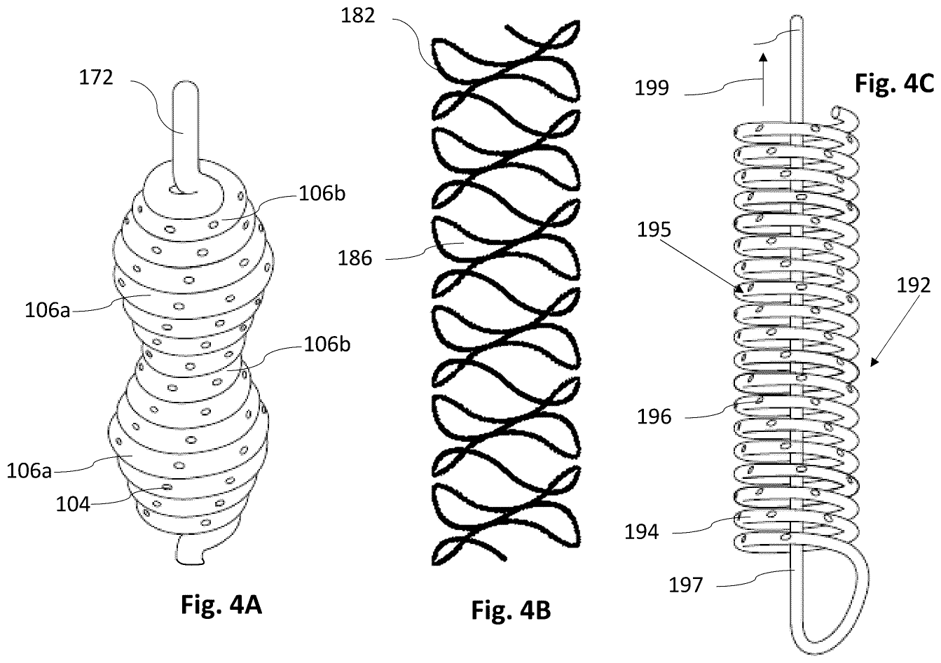

Reference is now made to FIGS. 4A, 4B, and 4C, which are schematic illustrations of exemplary structures of a coil formed of an elongate tube forming part of the medical system of FIG. 1, according to embodiments of the disclosed technology.

FIG. 4A shows a coil formed of an elongate tube 172, which is similar in structure to elongate tube 102 of FIG. 1. Specifically, elongate tube 172 includes an internal channel (not explicitly shown), a plurality of orifices 104 distributed longitudinally along the tube, and forms a coil including a plurality of loops 106.

However, the coil formed by elongate tube 172 differs from the coil formed by elongate tube 102, in which all the loops have the same internal and external diameters (see FIG. 1). In the embodiment illustrated in FIG. 4A, loops 106 have multiple different diameters. Additionally, some loops having the same diameter, are not adjacent one another. For example, FIG. 4A shows a first pair of loops 106a have a first, and largest, diameter, disposed at a distance dl from one another. A second pair of loops 106b have a second diameter, smaller than the first diameter of loops 106a, and are disposed at a second distance d2 from one another.

Varying loop diameters can form many different configurations, all of which are considered within the scope of the disclosed technology. For example, in some embodiments, and as shown in FIG. 4A, loops having different diameters may cause the coil to have an exterior shape similar to that of a spindle or an hourglass. In other embodiments, loops having varying diameters may form a coil having a generally conical or frusto-conical structure. In yet other embodiments, the loops of the coil may alternate between loops having a first, larger, diameter and loops having a second, smaller, diameter.

FIG. 4B shows a coil formed of an elongate tube 182, which is similar in structure to elongate tube 102 of FIG. 1. Specifically, elongate tube 182 includes an internal channel (not explicitly shown), a plurality of orifices (not explicitly shown) distributed longitudinally along the tube, and forms a coil including a plurality of loops 186. However, each of loops 186 has an at least partially sinusoidal structure. As such, space is created between each adjacent pair of loops 186. The at least partially sinusoidal structure shown in FIG. 4B is adapted to maintain the space between pairs of adjacent loops 186 when negative pressure is applied to elongate tube 182, and consequently to maintain the length of the coil when negative pressure is applied thereto. Consequently, in some embodiments, the length L2 of the coil in the draining operative state is substantially similar to the length L of the coil in the resting operative state.

In the context of the present application, a loop has a “sinusoidal structure” if the loop is not flat and is undulating (in the theta direction when revolving around the center of the loop). Stated differently, a loop has a “sinusoidal structure” if in at least one loop (i.e. 360-degree trip in the theta direction), or in each loop, there is are multiple longitudinal direction changes in the loop (e.g., from going up to going down to going back down) or multiple changes in the sign of the slope of the loop. As such, if an ant were to walk along the loop, it would change from walking “uphill” to walking “downhill” multiple times along a single loop, or along each loop.

FIG. 4C shows a coil formed of an elongate tube 192, which is similar in structure to elongate tube 102 of FIG. 1. Specifically, elongate tube 192 includes an internal channel (not explicitly shown), a plurality of orifices 194 distributed longitudinally along the tube, and includes a coil portion 195 forming a coil including a plurality of loops 196. However, elongate tube 192 further includes a linear portion 197, extending from an end 198 of coil portion 195 toward the fluid-tight lumen 108 (see FIG. 1) as indicate by arrow 199. Linear portion 197 extends through a longitudinal center of the coil. When linear portion 197 is coupled to fluid-tight lumen 108 and to the source of negative pressure, negative pressure delivered to the elongate tube 192 from fluid-tight lumen 108 traverses linear portion 197 of the elongate tube before reaching coil portion 195 and being delivered to the vicinity of the coil via orifices 194. Linear portion 197 extending from the distal end of the coil, through the center of coil portion 195, and being connected to fluid tight lumen 108, may be advantageous in maintaining the length of the coil from extending down the gastrointestinal tract, or from extending the pitch between loops 196 of the coil, when the coil is deployed in the gastrointestinal tract and negative pressure is applied thereto.

The following description relates to elongate tubes 102, 122, 132, 142, 152, 162, 172, 182, and 192 shown in FIGS. 1-4C. While the description uses the reference numerals provided with respect to FIG. 1, it is to be appreciated that it is similarly applicable to the same elements of FIGS. 2A to 4C, even if those elements are designated by different reference numerals.

In some embodiments, elongate tube 102 has a diameter d, shown in FIG. 1, in the range of 1 mm to 8 mm, 1 mm to 5 mm, or 2 mm to 4 mm.

In some embodiments, elongate tube 102 may be formed of a porous material, such as ePTFE, PTFE-foam, EVA, PU-foam, and PP-foam. In some embodiments, the orifices 104 may be or may include pores in the porous material.

some embodiments, elongate tube 102 may comprise, or may consist of, a radiopaque marker, radioactive marker, magnetic marker, and/or magnetic resonance marker. In some embodiments, elongate tube 102 may comprise, or may consist of, a metal, a natural or elastic polymer, a plastic, a shape memory alloy, a super clastic alloy, a biocompatible material, a biodegradable material, a bioresorbable material, and/or a bioabsorbable material.

In some embodiments, elongate tube 102 may be formed of a material relatively unlikely to irritate the gastrointestinal tract.

In some embodiments, elongate tube 102 may configured for an antimicrobial or anti-inflammatory effect. In some embodiments, elongate tube 102 comprises an antimicrobial or anti-inflammatory material. In some embodiments, elongate tube 102 is pretreated or coated with an antimicrobial or anti-inflammatory agent.

In some embodiments, elongate tube 102 is adapted to deliver to the vicinity of wound 12 an antimicrobial or anti-inflammatory medicament for treatment of the wound. For example, this may be accomplished by delivery of a medicament fluid via the channel and orifices of the elongate tube, as described hereinabove.

In some embodiments, elongate tube 102 has a textured exterior surface adapted to frictionally engage an interior surface of the gastrointestinal tract. An example of such a textured exterior surface is illustrated in FIGS. 3A to 3C, and its advantages are described hereinabove.

In some embodiments, elongate tube 102 may comprise, or may consist of, a shape memory material, an elastic material, a super-elastic material, or another polymeric material, adapted to direct or result in formation of a predetermined coil shape. In some embodiments, the predetermined coil shape has a longitudinal resilience, despite having a degree of collapsibility and expandability, in the first, resting operative state. It is understood that various mechanical properties of the material(s) used to form elongate tube 102, including brittleness, ductility, elasticity, hardness, malleability, plasticity, strength, and toughness, may be suitably selected to direct formation of the coil shape.

In some embodiments, the elongate tube 102 may have any suitable cross-sectional shape, including a circular cross section, an oval cross section, a D-shaped cross section, an I-shaped cross section, or a rectangular cross section.

In some embodiments, elongate tube 102 may have embedded therein one or more wires, such as wires 128, 138, 148, or 158 shown and described with respect to FIGS. 2A to 2E.

As seen from comparison of FIGS. 2A to 2E, the channel(s) within elongate tube 102 have any suitable shape or cross section. For example, the channel(s) may have a circular cross section or a polygonal cross section in a direction perpendicular to a longitudinal axis of the elongate tube. In embodiments in which elongate tube 102 includes more than one channel, the channels need not have the same cross section, in a direction perpendicular to the longitudinal axis of the elongate tube, as seen clearly in FIGS. 2B and 2D.

In some embodiments, the cross-sectional shape of the elongate tube, of the wire, and/or of the channel(s) may be selected to improve mechanical characteristics of the elongate tube to the functionality for which it is used. In some embodiments, the cross-sectional shape of the elongate tube, of the wire, and/or of the channel(s) may be selected to reduce deformation of the elongate tube, and to increase repeatability on cycle hysteresis. In some embodiments, the cross-sectional shape of the elongate tube, of the wire, and/or of the channel(s) may be selected to reduce plastic deformation at high strains. In some embodiments, the cross-sectional shape of the elongate tube, of the wire, and/or of the channel(s) may be selected to facilitate thermoforming of the tube, as described herein. In some embodiments, the cross-sectional shape of the elongate tube, of the wire, and/or of the channel(s) may be selected to reduce elongation of the elongate tube during pushing and/or pulling thereof.

In some embodiments, the composition and shape of the wire, or coil frame, is adapted to provide reinforcement to maintain the size and shape of the coil. The composition and shape of the coil frame may also provide flexibility to the loops, and to the coil as a whole, to permit stretching and compressing of the coil while preventing formation of kinks. In some embodiments, the wire comprises a material having a thermal-shaping temperature which does not substantially affect the elongate body structure. In some embodiments, the thermo-shaping temperature is lower than a melting point of a material from which the elongate tube body is formed.

In some embodiments, orifices 104 are disposed about a single circumference of the elongate tube, for example as shown in FIGS. 3A and 3B. In some embodiments, orifices 104 can be disposed along a single longitudinal position along the longitudinal axis of elongate tube 102 (as shown in FIG. 1), such that when the elongate tube is uncoiled, the orifices form a straight line along the length of the tube. In some embodiments, orifices 102 may be disposed along multiple longitudinal positions along the longitudinal axis of the elongate tube.

In some embodiments, at least one longitudinal channel extends along an external longitudinal portion of the elongate tube, as shown in FIGS. 3A and 3B.

In other embodiments, orifices 104 are disposed longitudinally, along a longitudinal length of elongate tube 102, or at different longitudinal positions along the longitudinal axis.

In some embodiments, and as shown in FIG. 1, orifices 104 may be equidistantly distributed along or about elongate tube 102. In other embodiments, orifices 104 may be heterogeneously distributed along or about elongate tube 102. For example, a first pair of adjacent orifices 104 may have a first longitudinal distance therebetween, and a second pair of adjacent orifices 104 may have a second longitudinal distance therebetween, the second longitudinal distance being different from the first longitudinal distance. As another example, a first pair of adjacent orifices 104 may have a first circumferential distance therebetween, and a second pair of adjacent orifices 104 may have a second circumferential distance therebetween, the second circumferential distance being different from the first circumferential distance.

In some embodiments, each of orifices 104 has substantially the same diameter. In some other embodiments, orifices 104 in a first subset of the orifices have a first diameter, and orifices 104 in a second subset of the orifices have a second diameter, the second diameter being different from the first diameter.

In the embodiment, the cross-sectional area of the orifices 104 increases along the length of the elongate tube, or of the coil, from the proximal end towards the distal end. In some such embodiments, the cross-sectional area of the distal-most orifice is at least 50% greater than the cross-sectional area of the proximal-most orifice.

In some embodiments, a greatest dimension of each of orifices 104 is within the range of 0.5 mm to 10 mm, 0.5 mm to 8 mm, 0.5 mm to 5 mm, 0.5 mm to 3 mm, or 1 mm to 2 mm.

In some embodiments, in the resting operative state of elongate tube 102, at least some of orifices 104 are oriented inwardly, toward a center of the coil, for example as shown in FIG. 3A. In some embodiments, in the resting state of elongate tube 102, at least some of orifices 104 are oriented outwardly, away from the center of the coil, for example as shown in FIG. 1.

In some embodiments, in the resting operative state, elongate tube 102 is substantially devoid of orifices 104 oriented outwardly, away from a center of the coil, such that all of orifices 104 are oriented toward the center of the coil.

In some embodiments, in the first, resting operative state of elongate tube 102, a length of the coil, indicated in FIG. 1 by L, is at least 10 mm, at least 15 mm, at least 20 mm, at least 25 mm, at least 30 mm at least 35 mm, at least 40 mm, at least 50 mm, at least 60 mm, or at least 80 mm.

In some embodiments, in the first, resting operative state of elongate tube 102, length L of the coil is at most 200 mm, at most 150 mm, at most 100 mm, at most 80 mm, at most 70 mm, at most 60 mm, or at most 50 mm.

In some embodiments, in the first, resting operative state of elongate tube 102, length L of the coil is in the range of 10 mm to 200 mm, 10 mm to 150 mm, 10 mm to 120 mm, 10 mm to 100 mm, 10 mm to 80 mm, 10 mm to 70 mm, 10 mm to 60 mm, 10 mm to 50 mm, 20 mm to 50 mm, 30 mm to 50 mm, 40 mm to 50 mm, 10 mm to 40 mm, 15 mm to 40 mm, 10 mm to 35 mm, or 15 mm to 35 mm.

In some embodiments, elongate tube 102 has a second, draining operative state, when negative pressure, for example in the range of 25-350 mmHg or 50-350 mmHg, is applied to the elongate tube. In the second draining operative state, the coil has a length L2, which is not greater than L. In some embodiments, in the draining operative state, the coil has a second length L2 in the range of 10 mm to 50 mm, 10 mm to 40 mm, 20 mm to 50 mm, or 20 mm to 40 mm.

In some embodiments, during application of negative pressure to elongate tube 102 and to the coil formed thereby, rings 106 of the coil tilt relative to the longitudinal axis of the coil (indicated by reference numeral 107 in FIG. 1). Such tilting of the rings 106 causes the cross-sectional area of each ring to change, for example from having a circular cross-sectional area to having an oval cross-sectional area. In some embodiments, each of rings 106 tilts, to the right or to the left, by up to 50 degrees, up to 45 degrees, up to 30 degrees, or up to 20 degrees From longitudinal axis 107. Typically, all the rings tilt in the same direction, and remain substantially parallel to each other. It is a particular feature of the disclosed technology that even when the rings 106 tilt relative to longitudinal axis 107, the coil remains distributed about the same longitudinal axis.

In some embodiments, a L:L2 ratio between length L of the coil in the resting operative state, and length L2 of the coil in the draining operative state is in the range of 1:1 to 4:1, 1:1 to 3:1, 1:1 to 2:1, 1:1 to 1.5:1, or 1:1 to 1.25:1 as measured in an ex-vivo female pig esophagus, of a pig weighing approximately 60 kg or in an in vitro model thereof.

In some embodiments, a difference between the cross-sectional diameter D of the coil, or in the cross sectional area of the coil, in the resting operative state and the second, draining, operative state, when negative pressure, for example in the range of 25-350 mmHg or 50-350 mmHg, is applied to elongate tube 102, is less than 75%, less than 50%, less than 20%, or less than 10%, of the cross-sectional diameter D or the cross sectional area in the resting operative state, as measured in an ex-vivo pig esophagus, of a pig weighing 60 kg or in an in vitro model thereof.

In some embodiments, a difference between length L of the coil in the resting operative state, and length L2 of the coil in the draining operative state is not greater than 150 mm, not greater than 125 mm, not greater than 125 mm, not greater than 100 mm, not greater than 75 mm, not greater than 50 mm, not greater than 40 mm, not greater than 30 mm, not greater than 20 mm, or not greater than 10 mm.

In some embodiments, in the first, resting, operative state of elongate tube 102, the coil has a uniform pitch between each pair of adjacent loops, for example as seen in FIGS. 1 to 3B.

In some embodiments, in the first operative state, a pitch of the coil is in a range of 2.5 mm to 25 mm. In some embodiments, in the first operative state, a pitch of the coil is in a range of 5 mm to 25 mm. In some embodiments, in the first operative state, a pitch of the coil is in a range of 2 mm to 40 mm. In some embodiments, in the first operative state, a pitch of the coil is in a range of 2 mm to 30 mm. In some embodiments, in the first operative state, a pitch of the coil is in a range of 2 mm to 25 mm. In some embodiments, in the first operative state, a pitch of the coil is in a range of 2 mm to 20 mm. In some embodiments, in the first operative state, a pitch of the coil is in a range of 3 mm to 15 mm.

In some embodiments, in the first, resting, operative state of elongate tube 102, the coil has a first pitch P. In some embodiments, in the second, draining, operative state of elongate tube 102, the coil has a second pitch P2. In some embodiments, a ratio between first pitch P and second pitch P2 is in the range of 1:1 to 6:1.

In some embodiments, the pitches P and/or P2 of the coil facilitate contraction and expansion of the coil in response to contraction and expansion of the gastrointestinal tract in which the coil is positioned, such that a three-dimensional position of the coil within the gastrointestinal tract, is maintained during motion of the gastrointestinal tract. For example, the position of the coil may be maintained to motion within the gastrointestinal tract, even during peristaltic contractions of the f.

In some embodiments, at least one characteristic of the coil is configurable by making a change to a condition in an environment surrounding the coil. In some embodiments, the characteristic may be, or may include, a chemical characteristic or a mechanical characteristic of the coil.

For example, the changed condition may be a temperature of the coil, or a temperature in the vicinity of the coil during the deployment.

In some embodiments, in the first, resting state of elongate tube 102, a number of loops 106 in the coil formed from the elongate tube is at least 3, at least 4, at least 5, at least 8, or at least 10.

In some embodiments, in the first, resting operative state of elongate tube 102, the number of loops 106 in the coil is within the range of 3 to 15, 5 to 15, 5 to 12, 8 to 12, 3 to 30, 5 to 30, 5 to 30, or 8 to 30.

In some embodiments, in the first, resting operative state of elongate tube 102, the diameter of each loop 106 in the coil or the diameter of the coil as a whole, indicated by D in FIG. 1, is in a range of 0.5 cm to 5 cm, 1 cm to 5 cm, 0.5 cm to 4 cm, 1 cm to 4 cm, 2 cm to 4 cm, 0.5 cm to 3 cm, 1 cm to 3 cm, 1 cm to 2.5 cm, 1 cm to 2 cm or 2 cm to 3.5 cm.

In some embodiments, in the first, resting operative state of elongate tube 102, the diameter of at least one of loops 106, and in some embodiments of each loop 106, in the coil is not greater than 1.5 cm.

In some embodiments, and as shown in FIG. 1, all of loops 106 have substantially the same external diameter.

In some embodiments, at least two loops 106 have substantially the same diameter. In some embodiments, the two loops having substantially the same diameter are a proximal-most loop, and a distal-most loop of the coil. In some embodiments, the diameter of loops 106 other than the proximal-most loop and the distal-most loop is not greater than the diameter of the proximal-most loop. In some embodiments, the diameter of loops 106 other than the proximal-most loop and the distal-most loop is not smaller than the diameter of the proximal-most loop.

In some embodiments, at least a subset of loops 106 are adapted to apply pressure, in a radial direction, to an interior surface of the gastrointestinal tract, such as to the interior surface of esophagus 10.

In some embodiments, when negative pressure is applied to elongate tube 102, loops 106 form a stack, which provides mechanical strength to the coil during application of the negative pressure.

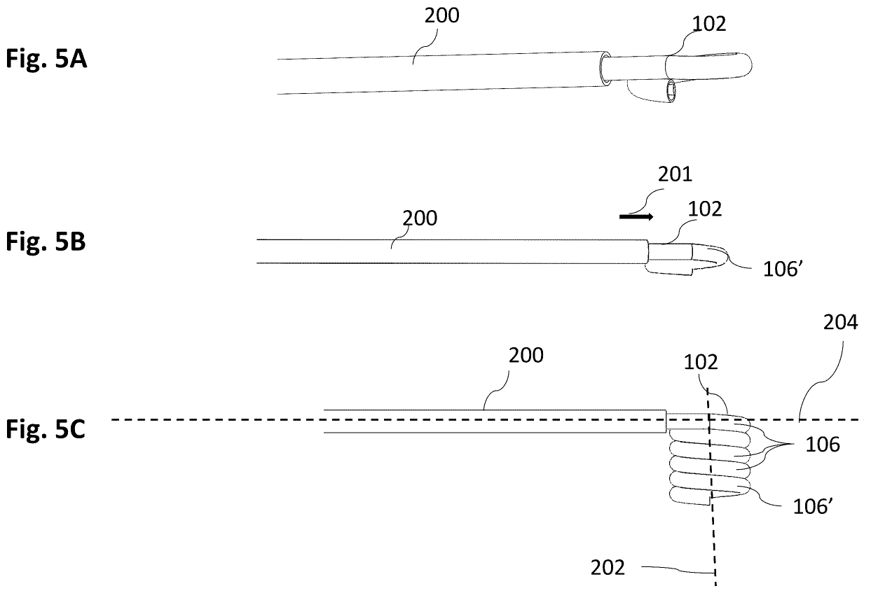

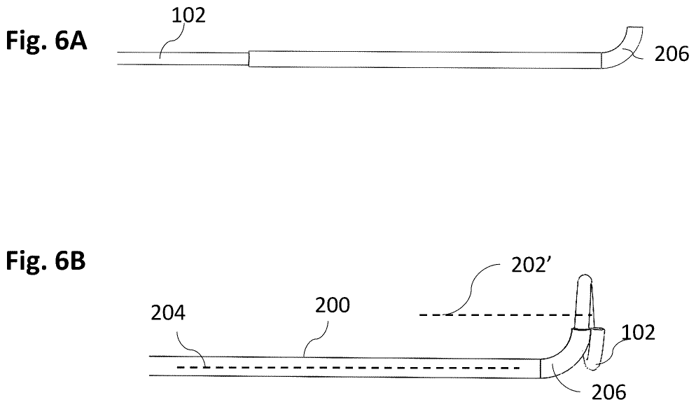

Reference is now made to FIGS. 5A, 5B and 5C, which are schematic illustrations of steps of deploying the medical system 100 into the gastrointestinal tract, for example into esophagus 10 according to an embodiment of the disclosed technology, and to FIGS. 6A and 6B, which are schematic illustrations of a modification of the steps of FIGS. 5A to 5C.

As seen in FIG. 5A, elongate tube 102 is disposed within a lumen of a tubular sheath 200, which functions as a delivery mechanism for delivering elongate tube 102 into the gastrointestinal tract. As such, in the embodiment of FIGS. 5A to 6B, elongate tube 102 is delivered into the gastrointestinal tract of the subject within the lumen of tubular sheath 200. In the embodiment of FIGS. 5A to 6B, elongate tube 102 may be associated with a wire (similar to wire 128, 138, 148, or 158 of FIGS. 2A to 2E) which is delivered into the gastrointestinal tract of the subject within the lumen of tubular sheath 200. In this case, the wire is adapted to act as a coil shaped frame for the elongate tube 102 when disassociated from the tubular sheath 200.

Elongate tube 102 is in a delivery state when associated with the tubular sheath 200 and obtains the resting operative state, in which the elongate tube is coiled, when dissociated from the sheath. Tubular sheath 200 is adapted to be removed from the gastrointestinal tract following delivery of elongate tube 102.

Tubular sheath 200 functions as a linearizing element for elongate tube 102, such that, in the delivery state, when the elongate tube is within sheath 200, elongate tube 102 is substantially linear and adapted for delivery, for example via a lumen of a working channel.

In some embodiments, elongate tube 102 is adapted to be removed from the gastrointestinal tract via the lumen of tubular sheath 200. In such embodiments, elongate tube 102 is pulled into the tubular sheath from the proximal end of the tube, and assumes the delivery state during removal thereof from the gastrointestinal tract.

In some embodiments, during deployment of the elongate tube, elongate tube 102 is adapted to form the coil sequentially as segments of the elongate tube are pushed distally out of tubular sheath 200, in the direction of arrow 201, as clearly seen by comparison of FIGS. 5B and 5C. As seen in FIGS. 5B and 5C, elongate tube 102 is pushed distally out of sheath 200, such that the distal end of the elongate tube forms the first, distal loop 106′, shown in FIG. 5B. Subsequently, as additional portions of elongate tube 102 are pushed out the sheath, they form additional loops 106, as shown in FIG. 5C.

In an inverse manner, during removal of elongate tube 102 from the gastrointestinal tract via sheath 200, each of loops 106 is adapted to transform into a substantially linear segment upon entry into the sheath, sequentially, from the proximal loop to the distal loop.

In some embodiments, a distal end of tubular sheath 200 includes a pointed shape. For example, the pointed shape may be desireable in order to pass through debris or through a wound scab on the way to a target destination of the elongate tube.

As seen in FIG. 5C, in some embodiments, a longitudinal axis 202 of the coil is angled (e.g. at a 90-degree angle) with respect to a longitudinal axis 204 of a remainder of elongate tube 102, for example disposed within sheath 200, or coupled (e.g., via the fluid-tight lumen) to the source of negative pressure 110 (FIG. 1). In some embodiments it is desirable for the longitudinal axes 202 and 204 to be parallel.

In some embodiments, and as shown in FIGS. 6A and 6B, tubular sheath 200 may have a shoulder 206 disposed at a distal end thereof. Shoulder 206 is adapted to redirect elongate tube 102 prior to coiling thereof. As such, and as shown in FIG. 6B, when the coil is formed, longitudinal axis 202′ of the coil is parallel to longitudinal axis 204 of the linear portion of sheath 200, which houses the linear remainder of elongate tube 102.

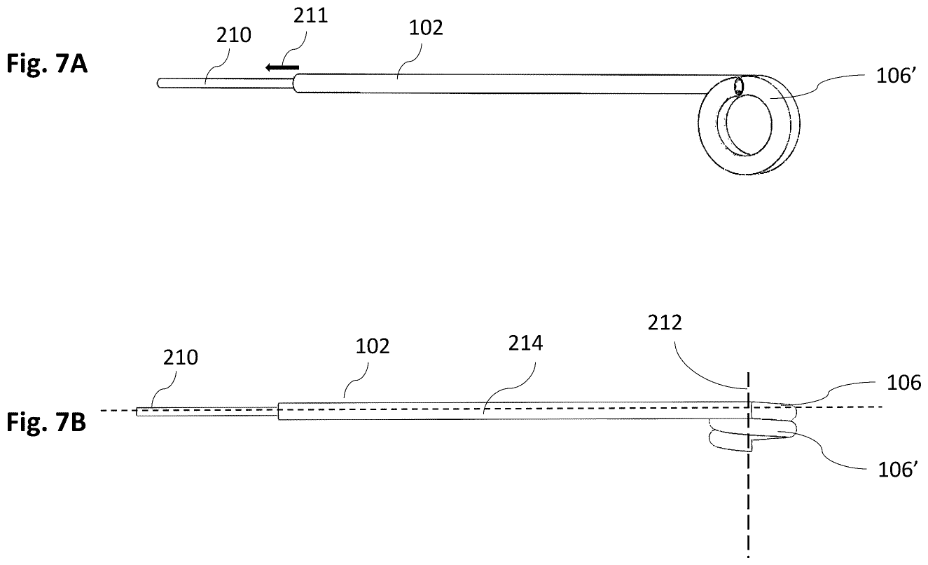

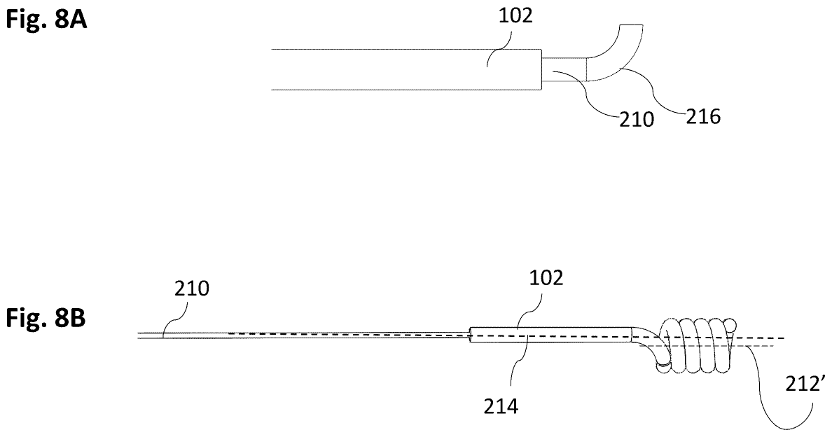

Reference is now made to FIGS. 7A and 7B, which are schematic illustrations of steps of deploying the medical system 100 into the gastrointestinal tract, for example into esophagus 10, according to an embodiment of the disclosed technology, and to FIGS. 8A and 8B, which are schematic illustrations of a modification of the steps of FIGS. 7A to 7B.

As seen in FIG. 7A, a wire 210, which may be a guidewire, is disposed within a channel of elongate tube 102, such as channel 125 (FIG. 2A). Wire 210 is adapted to be delivered into the gastrointestinal tract together with elongate tube. As such, in the embodiment of FIGS. 7A to 8B, elongate tube 102 is delivered into the gastrointestinal tract of the subject with wire 210 extending longitudinally and internally therethrough. In some embodiments, wire 210 may extend through a channel within elongate tube, distinct from the channel used for connection to the source of negative pressure 110 (FIG. 1). For example, wire 210 may extend through a flushing channel or through a dedicated channel.

In some embodiments, elongate tube 102 is in a delivery state when associated with wire 210 and obtains the resting operative state, in which the elongate tube is coiled, when dissociated from the wire. Wire 210 is adapted to be removed from the gastrointestinal tract following delivery of elongate tube 102, by pulling the wire in the direction of arrow 211, shown in FIG. 7A. In this case, wire 210 functions as a linearizing element for elongate tube 102, such that, in the delivery state, when wire 210 is within the elongate tube, elongate tube 102 is substantially linear. In some embodiments, elongate tube 102 is adapted to form the coil sequentially as segments of wire 210 are extracted proximally out of the elongate tube in the direction of arrow 211, as clearly seen by comparison of FIGS. 7A and 7B. For example, elongate tube 102 may include a coiling wire, similar to wires 128, 138, 148, and 158 of FIGS. 2A to 2E, which extends through or is embedded within the elongate tube and is adapted to form the coil. As seen in FIGS. 7A and 7B, guidewire 210 is pulled proximally out of elongate tube 102, such that the distal end of the elongate tube forms the first, distal loop 106′, shown in FIG. 7A. Subsequently, as additional portions of elongate tube 102 are dissociated from wire 210 which is pulled out of those portions, they form additional loops 106, as shown in FIG. 7B.

As seen in FIG. 7B, in some embodiments, a longitudinal axis 212 of the coil is angled (e.g. at a 90-degree angle) with respect to a longitudinal axis 214 of a remainder of elongate tube 102, for example having wire 210 disposed therein, or connected to the source of negative pressure 110 (FIG. 1). In some embodiments it is desirable for the longitudinal axes 212 and 214 to be parallel.

In some embodiments, and as shown in FIGS. 8A and 8B, wire 210 may have a shoulder 216 at a distal end thereof. Shoulder 216 is adapted to redirect elongate tube 102 prior to coiling thereof. As such, and as shown in FIG. 8B, when the coil is formed, longitudinal axis 212′ of the coil is parallel to longitudinal axis 214 of the linear portion elongate tube 102.

It is to be appreciated that, in some embodiments, elongate tube may be delivered into the gastrointestinal tract within a working channel of a delivery device. In some such embodiments, elongate tube 102 may be delivered within the working channel together with a linearizing clement, such as sheath 200 (FIGS. 5A to 6B) or guidewire 210 (FIGS. 7A to 8B). In such embodiments, elongate tube 102 (and the associated linearizing element) is sized and configured to pass through the working channel of the delivery device.

In some embodiments, the delivery device may be a catheter or an endoscope.

In some embodiments, the delivery device may include an image capturing element, adapted to provide images of elongate tube 102 during delivery thereof into the gastrointestinal tract.

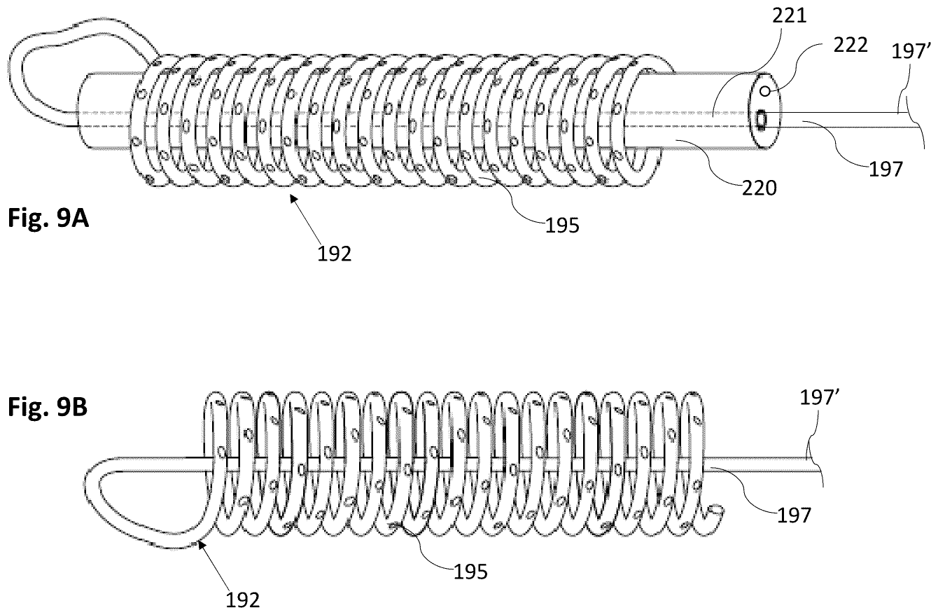

Reference is now made to FIGS. 9A and 9B, which are schematic illustrations of a method of deploying medical system 100 into the gastrointestinal tract for example into esophagus 10, using a delivery device 220, according to an embodiment of the disclosed technology. The method illustrated in FIGS. 9A and 9B results in the coil shape shown in FIG. 4C and described hereinabove. As such, the description below relates to the reference numerals shown in FIG. 4C.

As seen in FIG. 9A, elongate tube 192 is mechanically attached to an exterior of delivery device 220, during delivery thereof into the gastrointestinal tract. In some embodiments, and as shown in FIG. 9A, during delivery of the elongate tube into the gastrointestinal tract, coil portion 195 of elongate tube 192 is coiled about an exterior of delivery device 220, while linear portion 197 of the elongate tube extends through a working channel 221 of delivery device 220. As such, in the embodiment of FIGS. 9A and 9B, the delivery state of elongate tube 102 may be a coiled state or a partially coiled state. The end 197′ of linear portion 197 is adapted to be coupled to the fluid tight lumen 108.

In some embodiments, linear portion 197 extending through delivery device 220 may be used to modify the tension of the coil portion 195 about the exterior of the delivery device, or to modify the diameter of the loops of the coil portion about the exterior of the delivery device, prior to introduction of the delivery device into a bodily lumen.

As a result, following disconnection of the elongate tube 192, and specifically of coil portion 195, from delivery device 220, a diameter of the loops of coil portion 195 may change relative to a diameter maintained while the coil was wrapped around the exterior of the delivery device. For example, the diameter of loops 196 may increase following removal of coil portion 195 from delivery device 220.

As seen in FIG. 9B, once elongate tube 192 is delivered to the gastrointestinal tract and the coil is positioned at a desired location within the gastrointestinal tract (e.g., in the vicinity of a wound), the elongate tube is adapted to be disassociated from delivery device 220. As shown in FIG. 4C, following dissociation of elongate tube 192 from delivery device 220, linear portion 197 of the elongate tube extends through the coil formed by coil portion 195, typically through its center.

In some embodiments, delivery device 220 may be a catheter or an endoscope.

In some embodiments, delivery device 220 may include an image capturing element 222, adapted to provide images of the elongate tube during delivery thereof into the gastrointestinal tract. For example, image capturing element 222 may be a video camera adapted to capture images of the interior of the gastrointestinal tract during placement of the elongate tube therein.

In some embodiments, system 100 includes a handle portion (not explicitly shown) mechanically couplable to an end of fluid-tight lumen 108, which is far from elongate tube 102, and which typically remains extracorporeal during deployment of the elongate tube into the gastrointestinal tract. Manipulation of the handle portion, for example by pushing or turning thereof, results in distal motion of elongate tube 102. In some embodiments, the handle portion is adapted to be detached from fluid-tight lumen 108 following delivery of elongate tube 102 into the gastrointestinal tract.

In some embodiments, manipulation of the handle portion causes elongate tube 102 to transition from the delivery state to the coiled, resting operative state. In some embodiments, the transition of elongate tube 102 into the coil is sequential, such that each of loops 106 is adapted to form as the handle portion delivers elongate tube 102 into the gastrointestinal tract, sequentially from the distal loop to the proximal loop, in a similar manner to that shown in FIGS. 5A to 8B.

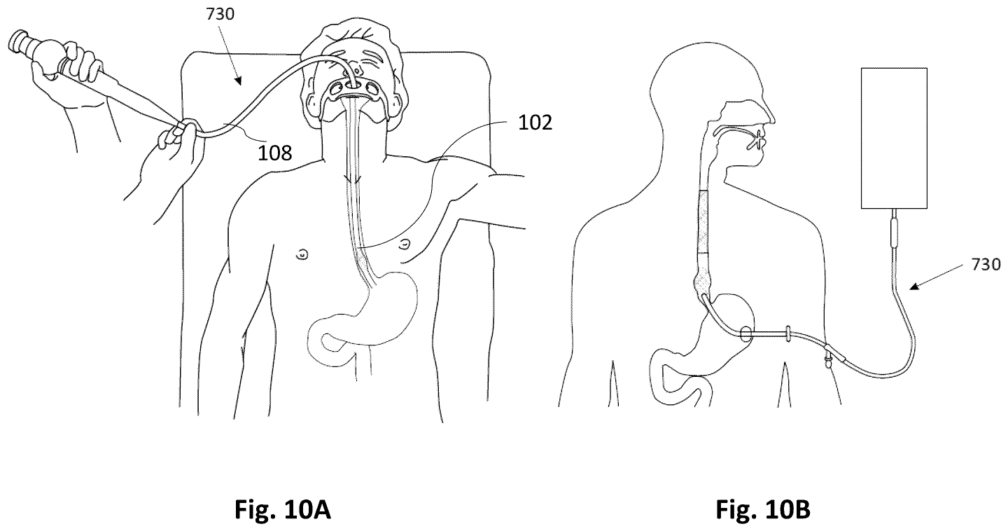

Reference is now made to FIGS. 10A and 10B, which are schematic illustrations of a procedure of deploying medical system 730, similar to medical system 100 including any one of elongate tubes 102, 122, 132, 142, 152, 162, 172, 182, or 192 into the body of a subject, according to embodiments of the disclosed technology.

As seen in FIG. 10A, system 730, and specifically the elongate tube thereof, may be fed into the gastrointestinal tract of the subject, via the subject's mouth. For example, in the illustrated embodiment, an endoscope is used to deliver the elongate tube into the esophagus of the subject, via the subject's mouth. However, it is to be appreciated that the method of FIGS. 10A and 10B may be used for delivery of the system to other parts of the gastrointestinal tract.

In FIG. 10B, the elongate tube of system 730 is delivered into the esophagus surgically. Specifically, a hole is punctured in the abdominal wall of the subject, and the elongate tube is delivered to the gastrointestinal tract, and specifically to the esophagus of the subject, via the stomach. Alternatively, a hole is punctured in the submental triangle (not shown) of the subject, and the elongate tube is delivered to the gastrointestinal tract and specifically to the esophagus of the subject via the punctured hole. In some such applications, a distal end of the elongate tube may be sharp, or may include a needle, suitable for puncturing the required hole in the abdominal wall. In some other embodiments, the process may be similar to that of placing a percutaneous endoscopic gastronomy (PEG) device, with the distinction that the elongate tube could be delivered further than the stomach, into the esophagus.

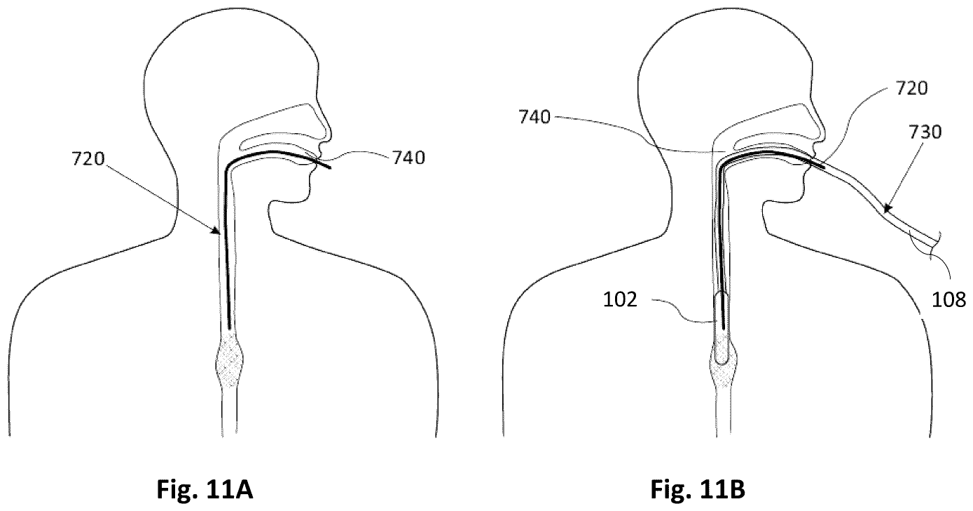

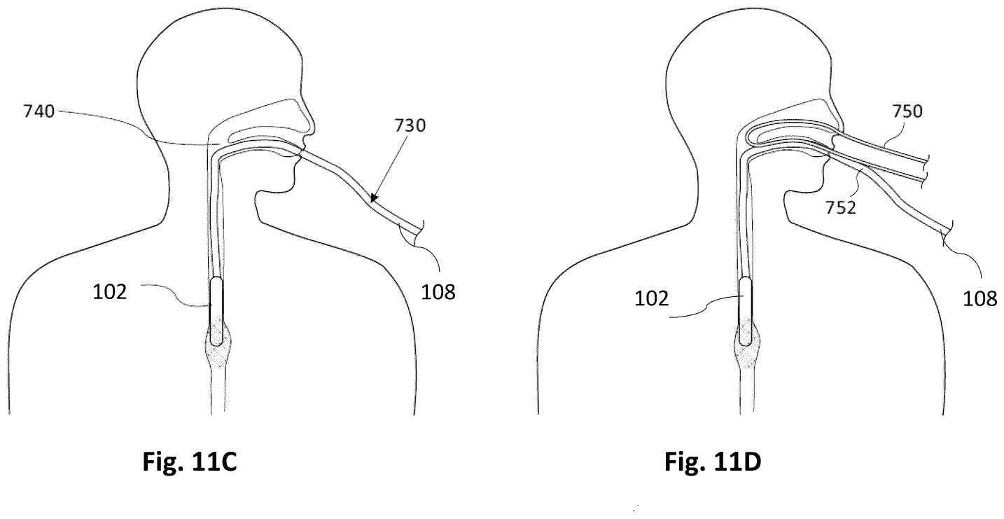

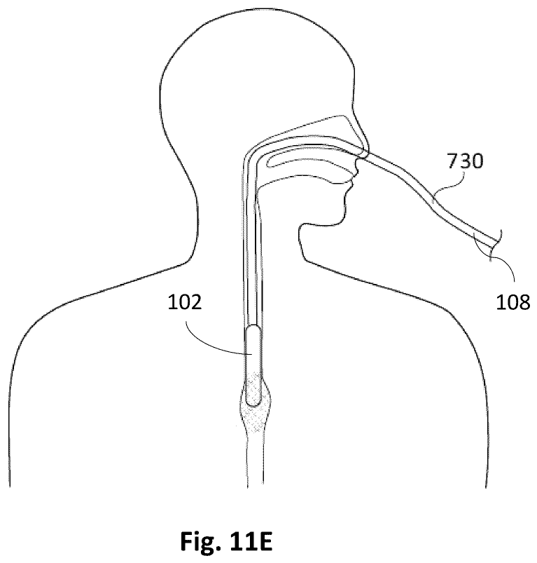

Reference is now made to FIGS. 11A, 11B, 11C, 11D, and 11E, which are schematic illustrations of steps of a procedure for maintaining medical system 730, such as medical system 100 including any one of elongate tubes 102, 122, 132, 142, 152, 162, 172, 182, or 192, in the body of the subject via a nasal wire or tube, following its introduction as shown in FIG. 10A. For brevity, the description relates to elongate tube 102 of medical system 730, relating to the elongate tube of FIG. 1. However, any of the other elongate tubes described hereinabove can replace elongate tube 102. However, it is to be appreciated that the method of FIGS. 11A and 11E may be used for delivery of the system to other parts of the gastrointestinal tract.

As seen, in FIG. 11A a delivery device 720 is deployed into the esophagus of the subject, via the subject's mouth 740. For example, the delivery device may be delivery device 220 described hereinabove with respect to FIG. 9A. In FIG. 11B, at least part of fluid-tight lumen 108, as well as elongate tube 102, are delivered to the esophagus of the user using delivery device 720 (not shown). Elongate tube 102 obtains the coil of its resting operative orientation, and the delivery device is removed from the mouth of the subject, leaving elongate tube 102 and fluid-tight lumen 108, in place. Following placement of medical system 730 within the esophagus of the subject, delivery device 720 is removed from the body, as seen in FIG. 11C.

In FIG. 11D, a wire 750 is inserted into the nose of the subject, and through the sinuses of the subject into, and out of, the subject's mouth. A proximal end of fluid-tight lumen 108 is then associated with end 752 of wire 750 extending out of the subject's mouth. In FIG. 11E, wire 750 is pulled out of the subject's nose. As wire 750 is pulled out of the subject's nose, end 752 of the wire, together with the proximal end of fluid tight lumen 108, are pulled into the subject's nose. When wire 750 is fully removed from the subject's nose, fluid tight lumen 108 continues to extend through the subject's nose, connecting elongate tube 102 to the exterior of the subject's body, for connection to the source of negative pressure 110. Fluid tight lumen 108 can additionally be associated with a nasal retaining clement which is configured to maintain the longitudinal position of the coil within the subject.

### Additional Discussion of Manufacture and Materials

The following description relates to elongate tubes 102, 122, 132, 142, 152, 162, 172, 182, and 192 shown in FIGS. 1-4C. While the description uses the reference numerals provided with respect to FIG. 1, it is to be appreciated that it is similarly applicable to the same elements of FIGS. 2A to 4C, even if those elements are designated by different reference numerals.

Generally, elongate tube 102 may be soft to prevent puncture of surrounding tissue, while also having sufficient strength and rigidity to maintain the coiled shape. In some embodiments, the elongate tube 102 may be formed of a single component which simultaneously provides the desired characteristics. In other embodiments, the elongate tube 102 may be formed from two or more components having distinct characteristics. For example, the components may include an elongate tube body having a lumen and walls that define orifices, and a rigid elastic coil frame (e.g. wire 158 of FIG. 2E). In some embodiments, the coil frame may be positioned cross-sectionally off-center and towards the external wall of the elongate tube body, as shown in FIG. 2E.

In some embodiments, one or more of orifices 104 in elongate tube 102 may be formed by laser drilling or by mechanical punching one or more holes. Alternatively, orifices 104 may be formed simultaneously with elongate body 102, such as by molding the elongate body using a mold having appropriate extensions.

In some embodiments, elongate tube 102 may be integrally formed, such as by suitable molding or extrusion. In other embodiments, the elongate body may have a coil frame may be separately manufactured and subsequently assembled. In particular, forming the elongate body may include integrally forming two structures that are substantially aligned and adjoined along a longitudinal edge. Alternatively, the two structures may be separately formed and attached to each other, via a lumen in the elongate tube. Other methods of forming the elongate tube also may be employed.

In some embodiments, a separate construction and assembly approach is employed wherein forming the elongate tube involves forming an elongate tube body including walls that define orifices and having flexibility, and a coil frame lumen, separately forming a more rigid coil frame, and associating the elongate tube body with the coil frame. Associating the elongate tube body with the coil frame may comprise inserting or threading the coil frame into the coil frame lumen of the elongate tube. In some embodiments, a distal end of the coil frame may be blunt or covered by a smooth ball of increased cross section during insertion of the coil frame into the lumen. The ball may facilitate driving the coil frame through the coil frame lumen without puncturing the wall of the elongate tube. In some embodiments, the elongate tube body may be slightly compressed between two surfaces during the insertion of the coil frame. Compression of the elongate tube body elongates the opening into the coil frame lumen, facilitating easier loading thereof with the coil frame.

The separate construction and assembly may allow pushing or pulling on the coil frame, to control the formation of the coil structure or linearization of the elongate tube.

In some embodiments, the elongate tube may be formed integrally, such as via molding, over molding, reflow, sequential extrusion or co-extrusion, such that the elongate tube body with the retention frame are associated by being formed together. In some cases, the coil frame and elongate body may be formed of two distinct polymers, and may have a defined contact layer at an interface of the two polymers. In other cases, such as over-molding, the contact layer may be less defined after heating.

It should be understood that the use of “and/or” is defined inclusively such that the term “a and/or b” should be read to include the sets: “a and b,” “a or b,” “a,” “b.”

The various systems, devices, apparatuses, etc. in this disclosure can be sterilized (e.g., with heat, radiation, ethylene oxide, hydrogen peroxide, etc.) to ensure they are safe for use with patients, and the methods herein can comprise such sterilization of the associated system, device, apparatus, etc. Furthermore, the scope of the present disclosure includes, for some applications, sterilizing one or more of any of the various systems, devices, apparatuses, etc. in this disclosure.

The present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description. Further, the techniques, methods, operations, steps, etc. described or suggested herein can be performed on a living animal or on a non-living simulation, such as on a cadaver, cadaver gastrointestinal tract, simulator (e.g., with the body parts, tissue, etc. being simulated), etc.

Although the operations of some of the disclosed examples are described in a particular, sequential order for convenient presentation, it should be understood that this manner of description encompasses rearrangement, unless a particular ordering is required by specific language set forth above. For example, operations or steps described sequentially can in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms can vary depending on the particular implementation and are discernible by one of ordinary skill in the art.

1. A medical system for applying negative pressure within a gastrointestinal tract of a subject, the system comprising:
an elongate tube defining:
at least one interior channel along at least a longitudinal portion of the elongate tube;
a plurality of orifices in fluid communication with the at least one interior channel; and
at least one exterior channel extending longitudinally along an exterior surface of the elongate tube, at least one of the at least one exterior channel being in fluid communication with the at least one interior channel via at least one of the plurality of orifices,
the elongate tube having a delivery state, and a first operative state in which the elongate tube forms a coil including a plurality of loops; and
a fluid-tight lumen in fluid communication with an end of the elongate tube, the fluid-tight lumen adapted to couple to a source of negative pressure and to deliver negative pressure to the elongate tube,
wherein, in the first operative state of the elongate tube, a length of the coil is at least 15 mm and the plurality of loops includes at least 4 loops.

2. The medical system of claim 1, wherein the at least one interior channel comprises a first interior channel adapted for drainage of a fluid from the gastrointestinal tract, via the at least one exterior channel and the plurality of orifices, when negative pressure is applied to the elongate tube.

3. The medical system of claim 1, wherein the elongate tube comprises a shape memory material, an elastic material, or a super-elastic material adapted to form the coil in the first operative state.

4. The medical system of claim 1, wherein the elongate tube has a textured exterior surface adapted to frictionally engage an interior surface of the gastrointestinal tract.

5. The medical system of claim 1, wherein the at least one exterior channel comprises a plurality of exterior channels forming troughs on the exterior surface of the elongate tube and extending longitudinally therealong.

6. The medical system of claim 1, wherein the plurality of orifices are disposed about a single circumference of the elongate tube.

7. The medical system of claim 6, wherein the at least one exterior channel extends longitudinally along the exterior surface of the elongate tube between the orifices and a distal end of the elongate tube.

8. The medical system of claim 1, wherein the plurality of orifices are disposed longitudinally along a longitudinal length of the elongate tube.

9. The medical system of claim 1, wherein orifices in a first subset of said plurality of orifices have a first diameter, and orifices in a second subset of said plurality of orifices have a second diameter, the second diameter being different from the first diameter.

10. The medical system of claim 1, wherein, in the first operative state, at least some of the plurality of orifices are oriented outwardly, away from a center of the coil.

11. The medical system of claim 1, wherein, in the first operative state, at least some of the plurality of orifices are oriented inwardly, toward a center of the coil.

12. The medical system of claim 1, wherein, in the first operative state of the elongate tube, at least one of the plurality of loops has a sinusoidal structure.

13. The medical system of claim 1, further comprising a linearizing element, wherein the elongate tube has the delivery state, which is a linear state, when associated with the linearizing element, and the first operative state, in which the elongate tube forms the coil, when dissociated from the linearizing element.

14. The medical system of claim 1, wherein the fluid-tight lumen, and the elongate tube in the delivery state, are sized and configured to pass through a working channel of a delivery device, wherein the working channel has a diameter smaller than 5 mm.

15. The medical system of claim 1, wherein the delivery state of the elongate tube is a coiled state, and the elongate tube is adapted to be delivered into the gastrointestinal tract of the subject disposed about the exterior of a delivery device.

16. A medical system for applying negative pressure within a gastrointestinal tract of a subject, the system comprising:
an elongate tube defining at least one channel along at least a longitudinal portion thereof and including a plurality of orifices in fluid communication with the at least one channel the elongate tube having a delivery state and a first operative state in which the elongate tube forms a coil including a plurality of loops, at least some of the plurality of loops having a sinusoidal structure; and
a fluid-tight lumen in fluid communication with an end of the elongate tube, the fluid-tight lumen adapted to couple to a source of negative pressure and to deliver negative pressure to the at least one channel via the end of the elongate tube,
wherein in the first operative state of the elongate tube, a length of the coil is at least 15 mm and the plurality of loops includes at least 4 loops.

17. The medical system of claim 16, wherein the at least one channel is adapted for drainage of a fluid from the gastrointestinal tract, via the plurality of orifices, when negative pressure is applied to the elongate tube.

18. The medical system of claim 16, wherein the elongate tube comprises a shape memory material, an elastic material, or a super-elastic material adapted to form the coil in the first operative state.

19. The medical system of claim 16, wherein the elongate tube has a textured exterior surface adapted to frictionally engage an interior surface of the gastrointestinal tract.

20-27. (canceled)

28. A medical system for applying negative pressure within gastrointestinal tract of a subject, the medical system comprising:
an elongate tube defining at least one channel along at least a longitudinal portion thereof and including a plurality of orifices in fluid communication with the at least one channel, the elongate tube including:
a coil portion forming a coil having a plurality of loops; and
a linear portion extending through a longitudinal center of the coil portion; and
a fluid-tight lumen in fluid communication with the elongate tube, the fluid-tight lumen adapted to couple to a source of negative pressure and to deliver negative pressure to the coil portion of the elongate tube,
wherein the elongate tube is adapted to be delivered into the gastrointestinal tract of the subject with the coil portion disposed about the exterior of a delivery device and the linear portion extending through the interior of the delivery device,
wherein a length of the coil is at least 15 mm and the plurality of loops includes at least 4 loops.

29-43. (canceled)
